(19) United States
(12) Patent Application Publication
(10) Pub. No.: US 2022/0089616 A1
(43) Pub. Date: Mar. 24, 2022

(54) CARBORANE-BASED HISTONE DEACETYLASE (HDAC) INHIBITORS

(71) Applicant: The Regents of the University of California, Oakland, CA (US)

(72) Inventors: Alexander Spokoyny, Los Angeles, CA (US); Jessica K. Logan, Los Angeles, CA (US); Azin Saebi, Los Angeles, CA (US); Rafal Dziedzic, Los Angeles, CA (US); Pamela J. Kennedy, Los Angeles, CA (US)

(21) Appl. No.: 17/298,225

(22) PCT Filed: Dec. 4, 2019
(86) PCT No.: PCT/US19/64536

§ 371 Date: May 28, 2021

Related U.S. Application Data
(60) Provisional application No. 62/775,220, filed on Dec. 4, 2018.

(51) Int. Cl. C07F 5/02; C07C 211/38

(52) U.S. Cl. CPC C07F 5/02; C07C 211/38

(57) ABSTRACT

The present disclosure provides compounds and compositions capable of treating cancer, a disease of the central nervous system, and an inflammatory autoimmune disease, and methods of use thereof.

### CROSS-REFERENCE TO RELATED APPLICATIONS

This application claims the benefit of priority to U.S. Provisional Patent Application No. 62/775,220, filed Dec. 4, 2018, the contents of which are incorporated herein by reference in their entirety.

### STATEMENT REGARDING FEDERALLY SPONSORED RESEARCH OR DEVELOPMENT

This invention was made with Government support under Grant Number GM124746, awarded by the National Institutes of Health. The government has certain rights in the invention.

### BACKGROUND

Histone deacetylase (HDAC) proteins are involved in different diseases, making selectivity a crucial requirement for therapeutics. HDACs are enzymes that catalyze the removal of acetyl functional groups from the lysine residues of both histone and non-histone proteins. HDAC enzymes have 11 known human isoforms divided into four classes: Class 1 (1, 2, 3, 8), Class IIa (4, 5, 7, 9), Class IIb (6, 10), and Class IV (11). There is an unmet need to develop HDAC inhibitors that are selective for certain HDAC isoforms.

### SUMMARY

Provided herein are compounds, compositions, and methods useful in the treatment of cancer, a disease of the central nervous system, and/or an inflammatory autoimmune disease. In some embodiments, the methods comprise administering a compound disclosed herein or a pharmaceutically acceptable salt, ester, or prodrug thereof. In certain embodiments, the compositions disclosed herein further comprise a pharmaceutically acceptable carrier.

In certain aspects, the present disclosure provides a compound having the structure of Formula (I)

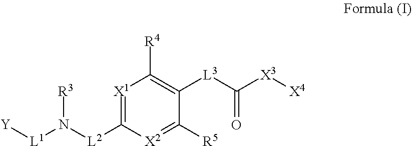

or a pharmaceutically acceptable salt, ester, or prodrug thereof, wherein
Y is optionally substituted boron cluster (e.g., an icosahedral boron cluster);
R³ is H or optionally substituted alkyl;
R⁴ is selected from H, halo, hydroxyl, and optionally substituted alkyl;
R⁵ is selected from H, halo, hydroxyl, and optionally substituted alkyl;
each of L¹, L², and L³ is independently selected from a bond, optionally substituted alkylene, and optionally substituted alkenylene;
X¹ is CR⁶ or N;
X² is CR⁷ or N;
each R⁶ and R⁷ is independently selected from H, halo, hydroxyl, and optionally substituted alkyl;
X³ is selected from —NH—, —N(OH)—, and O; and
X⁴ is selected from H, optionally substituted alkyl, and optionally substituted aryl.

In certain embodiments, the boron cluster is a B₁₂H₁₂, B₁₀H₁₀, or B₆H₆ cluster, or a carborane. In certain such embodiments, the boron cluster is a B₁₂H₁₂, B₁₀H₁₀, or B₆H₆ cluster. In other such embodiments, the boron cluster is a carborane.

In certain embodiments, the carborane is a C₂B₁₀ carborane, such as an icosahedral closo-carborane. In certain such embodiments, Y is selected from

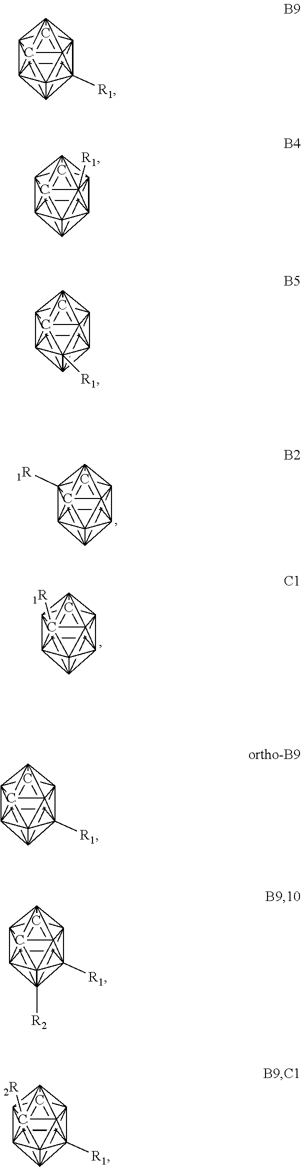

pharmaceutically acceptable salt thereof,
wherein
the unlabeled atoms of the icosahedron are boron;
R¹ represents a bond to L¹; and
R² is selected from halo, cyano, optionally substituted alkyl, optionally substituted amine, and —OR⁸, wherein R⁸ is optionally substituted alkyl or a protecting group.

In certain embodiments, R³ is alkyl or haloalkyl. In certain such embodiments, R³ is alkyl, preferably methyl.

In certain embodiments, each of R⁴, R⁵, R⁶, and R⁷ is independently selected from H, halo, hydroxyl, methyl, and —CF₃, such as wherein each of R⁴, R⁵, R⁶, and R⁷ is independently selected from H, —F, —Cl, and —Br, such as wherein R⁴, R⁵, R⁶, and R⁷ are each H.

In certain embodiments, L¹ and L² are each independently alkylene, such as wherein L¹ and L² are each independently —CH₂—.

In certain embodiments, L³ is alkenylene, such as —CH═CH—. In other embodiments, L³ is a bond.

In certain embodiments, X¹ is CR⁶. In some such embodiments, R⁶ is H.

In certain embodiments, X² is CR⁷, such as wherein R⁷ is H.

In certain embodiments, X³ is —N(OH)—. In certain embodiments, X⁴ is H.

In certain embodiments, the compound has the structure:

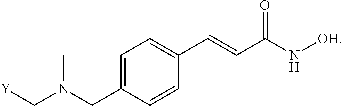

In certain embodiments, the compound is

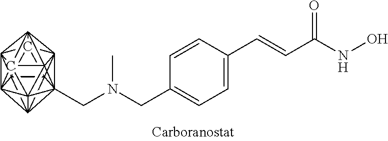

or a pharmaceutically acceptable salt thereof.

In certain embodiments, the compound has the structure:

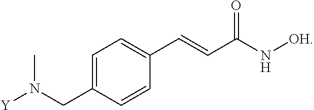

In certain embodiments, the compound is

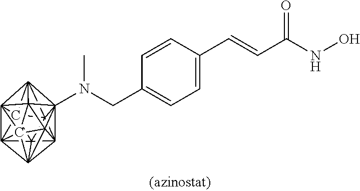

or a pharmaceutically acceptable salt thereof.

In some embodiments, the compounds disclosed herein have a structure as shown herein or a pharmaceutically acceptable salt, ester, or prodrug thereof.

In certain aspects, the present disclosure provides pharmaceutical compositions comprising a compound as disclosed herein and a pharmaceutically acceptable excipient.

In certain aspects, the present disclosure provides methods of inhibiting an histone deacetylase (HDAC) enzyme in a subject, comprising administering to the subject a compound or composition as disclosed herein.

In certain aspects, the present disclosure provides methods of treating a disease selected from a cancer, a disease of the central nervous system, or an autoimmune disease in a subject, comprising administering to the subject a compound or composition as disclosed herein. In certain embodiments, the disease is cancer, such as a cancer selected from glioma, e.g., glioblastoma; hematological cancer, e.g., leukemia or lymphoma; and non-small cell lung cancer. In other embodiments, the disease is a disease of the central nervous system, such as a disease of the central nervous system selected from mood and mental disorders, e.g., depression, schizophrenia, or bipolar disorder; neurodegenerative diseases, e.g., Huntington's disease, or Alzheimer's disease; drug addiction, e.g., cocaine addiction; and disorders of learning, memory, or cognition. In still other embodiments, the disease is an inflammatory autoimmune disease.

In certain aspects, the present disclosure provides processes for preparing a compound of Formula (I)

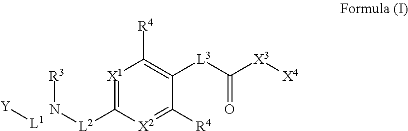

comprising:
preparing a compound of Formula (II) by reacting a compound of Formula (III) with a compound of Formula (IV) under reductive alkylation conditions:

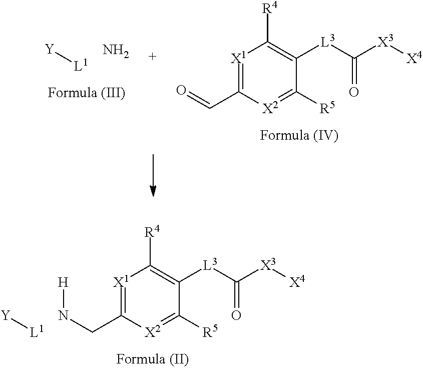

### BRIEF DESCRIPTION OF THE DRAWINGS

FIGS. 1A and 1B show ¹H NMR (FIG. 1A) and ¹³C NMR (FIG. 1B) spectra of adamantane carbonitrile (AV500, in CDCl3).

FIGS. 1C and 1D show ¹H NMR (FIG. 1C; AV400, in CDCl3) and ¹³C NMR (FIG. 1D; AV500, in CDCl3) spectra of adamantane methylamine.

FIG. 2A shows a 1H NMR spectrum of adamantane cinnamate imine (AV400, in CDCl3).

FIG. 2B shows a 1H NMR spectrum of adamantane cinnamate (AV400, in CDCl3), and FIG. 2C shows a 13C NMR spectrum of adamantane cinnamate (AV500, in CDCl3).

FIGS. 3A and 3B show 1H NMR (FIG. 3A; AV400, in CDCl3) and 13C NMR (FIG. 3B; AV500, in CDCl3) spectra of adamantane methyl cinnamate.

FIGS. 4A and 4B show 1H NMR (FIG. 4A; AV500, in DMSO-d6) and 13C NMR (FIG. 4B; AV500, in DMSO-d6) spectra of martinostat.

FIGS. 5A-5D show 1H NMR (FIG. 5A; AV500, in DMSO-d6), 13C NMR (FIG. 5B; AV500, in DMSO-d6), 11B NMR (FIG. 5C; AV400, in DMSO-d6), and 11B{1H} NMR (FIG. 5D; AV400, in DMSO-d6) spectra of azinostat.

FIG. 6 shows a schematic of a protein binding affinity assay described in Example 3A.

FIG. 7 shows HDAC2 inhibition data for carboranostat, martinostat, and azinostat.

FIG. 8A shows % live cells for CHO cells treated with 300 nM-40 μM carboranostat or martinostat. FIGS. 8B-8G show the raw FACS data for cells receiving no treatment (FIG. 8B), heat-killed cells (FIG. 8C), and cells treated with 300 nM carboranostat (FIG. 8D), 1 μM martinostat (FIG. 8E), 40 μM martinostat (FIG. 8F), or Triton X-100 9% (FIG. 8G).

FIGS. 9A and 9B show MTS assay data for CHO cells (FIG. 9A) and mouse NPCs (FIG. 9B) either untreated or treated with 300 nM-40 μM carboranostat or martinostat. Cells were plated at 10K and 20K cells/well in each group.

FIGS. 10A-10G show Green TUNEL assay data for CHO cells treated with 300 nM or 40 μM carboranostat or martinostat.

FIG. 11A shows TUNEL assay data for mouse NPCs treated with 300 nM or 40 μM carboranostat or martinostat. FIG. 11B shows representative confocal images.

### DETAILED DESCRIPTION

The present disclosure provides carborane-based HDAC inhibitors, which are preferably selective for one or more specific isoforms of the HDAC relative to other isoforms of HDAC inhibitors, e.g., isoforms that, when inhibited, can lead to undesirable side effects.

In certain aspects, provided herein are compounds, compositions, and methods useful in the treatment of cancer, a disease of the central nervous system, and/or an inflammatory autoimmune disease. In some embodiments, the methods comprise administering a compound disclosed herein or a pharmaceutically acceptable salt, ester, or prodrug thereof. In certain embodiments, the compositions disclosed herein further comprise a pharmaceutically acceptable carrier.

### I. Compounds

Disclosed herein are compounds comprising dicarba-closo-dodecaborane. Dicarba-closo-dodecaborane (a preferred carborane in the context of the compounds disclosed herein) is an icosahedral cluster containing two carbon atoms and ten boron atoms in which both carbon atoms are hexacoordinated. Depending on the position of the carbon atoms in the cluster, three kinds of isomers exist: 1,2-dicarba-closo-dodecaborane (ortho-carborane), 1,7-dicarba-closo-dodecaborane (meta-carborane), and 1,12-dicarba-closo-dodecaborane (para-carborane). These structures are unique among boron compounds, as they can have high thermal stabilities and hydrophobicities, for example, comparable to hydrocarbons.

In one aspect, provided herein are compounds having the structure of Formula (I)

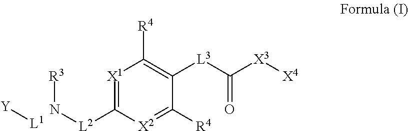

or a pharmaceutically acceptable salt, ester, or prodrug thereof, wherein
Y is optionally substituted boron cluster (e.g., an icosahedral boron cluster);
R³ is H or optionally substituted alkyl;
R⁴ is selected from H, halo, hydroxyl, and optionally substituted alkyl;
R⁵ is selected from H, halo, hydroxyl, and optionally substituted alkyl;
each of L¹, L², and L³ is independently selected from a bond, optionally substituted alkylene, and optionally substituted alkenylene;
X¹ is CR⁶ or N;
X² is CR⁷ or N;
each R⁶ and R⁷ is independently selected from H, halo, hydroxyl, and optionally substituted alkyl;
X³ is selected from —NH—, —N(OH)—, and O; and
X⁴ is selected from H, optionally substituted alkyl, and optionally substituted aryl.

In certain embodiments, the boron cluster is a B₁₂H₁₂, B₁₀H₁₀, or B₆H₆ cluster, or a carborane. In certain such embodiments, the boron cluster is a B₁₂H₁₂, B₁₀H₁₀, or B₆H₆ cluster. In other such embodiments, the boron cluster is a carborane.

In some embodiments, the compounds disclosed herein have a carborane structure selected from 1,5-C₂B₃H₅, 1,2-C₂B₄H₆, 1,6-C₂B₄H₆, 1-CB₅H₇, 2,3-C₂B₅H₇, 2,4-C₂B₅H₇, 1,7-C₂B₆H₈, 4,5-C₂B₇H₉, 1,2-C₂B₈H₁₀, 1,6-C₂B₈H₁₀, 1,10-C₂B₈H₁₀, 2,3-C₂B₉H₁₁, 1,2-C₂B₁₀H₁₂, 1,7-C₂B₁₀H₁₂, 1,12-C₂B₁₀H₁₂, 1,2-C₂B₃H₇, 2-CB₅H₉, 2,3-C₂B₄H₈, 2,3,4-C₃B₃H₇, 2,3,4,5-C₄B₂H₆, 6,8-C₂B₇H₁₃, 5,6-C₂B₈H₁₂, 5,6,7-C₃B₇H₁₃, 7,8-C₂B₉H₁₃ and 2,3,7,8-C₄B₈H₁₂.

In some embodiments, the compounds disclosed herein have a carborane structure selected from C₂B₃H₅, CB₃H₇, C₂B₄H₆, C₂B₃H₇, C₂B₅H₇, C₂B₄ H₈, CB₅H₉, CB₄H₁₀, C₂B₆H₈, C₂B₄H₁₀, C₂B₇H₉, C₂B₆H₁₀, C₂B₅H₁₁, C₂B₈H₁₀, C₂B₇H₁₁, C₂B₆H₁₂, C₂B₉H₁₁, C₂B₈H₁₂, C₂B₇H₁₃, C₂B₁₀H₁₂, C₂B₉H₁₃, C₂B₈H₁₄, C₃B₃H₇, C₄B₂H₆, C₃B₇H₁₃ and C₄B₈H₁₂.

In some embodiments, the compounds disclosed herein have a structure as shown herein or a pharmaceutically acceptable salt, ester, or prodrug thereof.

In certain embodiments, the carborane is a C₂B₁₀ carborane, such as an icosahedral closo-carborane. In certain such embodiments, Y is selected from

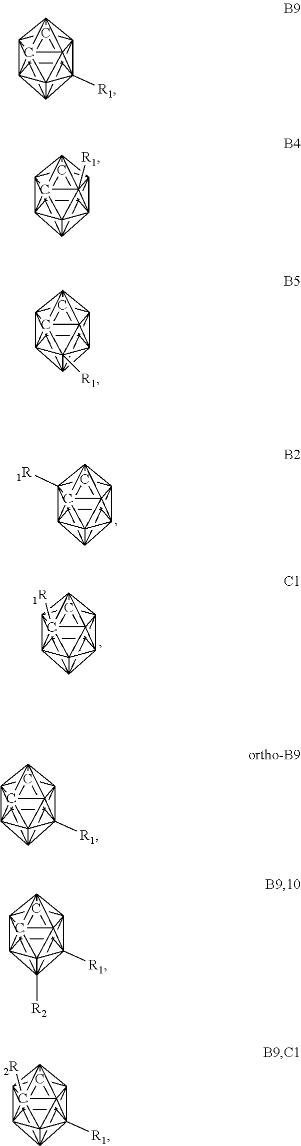

or a pharmaceutically acceptable salt thereof,
wherein
the unlabeled atoms of the icosahedron are boron;
R¹ represents a bond to L¹; and
R² is selected from halo, cyano, optionally substituted alkyl, optionally substituted amine, and —OR⁸, wherein R⁸ is optionally substituted alkyl or a protecting group. Substituents on the icosahedron follow standard organic chemical conventions (e.g., unlabeled atoms in the substituent represent carbon).

In certain embodiments, R³ is alkyl or haloalkyl. In certain such embodiments, R³ is alkyl, preferably methyl.

In certain embodiments, each of R⁴, R⁵, R⁶, and R⁷ is independently selected from H, halo, hydroxyl, methyl, and —CF₃, such as wherein each of R⁴, R⁵, R⁶, and R⁷ is independently selected from H, —F, —Cl, and —Br, such as wherein R⁴, R⁵, R⁶, and R⁷ are each H.

In certain embodiments, L¹ and L² are each independently alkylene, such as wherein L¹ and L² are each independently —CH₂—.

In certain embodiments, L³ is alkenylene, such as —CH═CH—. In other embodiments, L³ is a bond.

In certain embodiments, X¹ is CR⁶. In some such embodiments, R⁶ is H.

In certain embodiments, X² is CR⁷, such as wherein R⁷ is H.

In certain embodiments, X³ is —N(OH)—. In certain embodiments, X⁴ is H.

In certain embodiments, the compound has the structure:

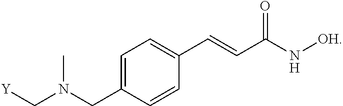

In certain embodiments, the compound is

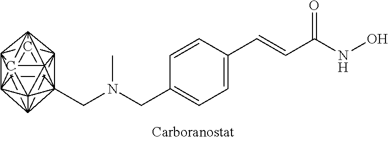

or a pharmaceutically acceptable salt thereof.

In certain embodiments, the compounds of the present disclosure have increased inhibition for various HDAC isoforms as shown herein.

In certain aspects, the present disclosure provides processes for preparing a compound of Formula (I)

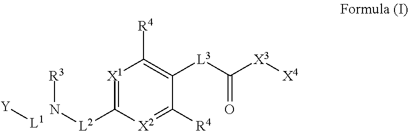

comprising:
preparing a compound of Formula (II) by reacting a compound of Formula (III) with a compound of Formula (IV) under reductive alkylation conditions:

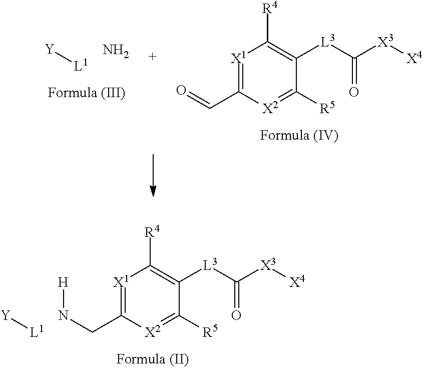

### II. Pharmaceutical Compositions

In certain aspects, the disclosure relates to a pharmaceutical composition comprising a compound described herein and a pharmaceutically acceptable carrier. In some embodiments, the disclosure relates to a pharmaceutical composition comprising any one of the aforementioned compounds and a pharmaceutically acceptable carrier.

Patients, including but not limited to humans, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

The concentration of active compound in the drug composition will depend on absorption, inactivation and excretion rates of the drug as well as other factors known to those of skill in the art. It is to be noted that dosage values will also vary with the severity of the condition to be alleviated, and possible drug-drug interactions with antiretroviral medications. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In certain embodiments, the mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose or sweetener as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, carriers include physiological saline and phosphate buffered saline (PBS).

In certain embodiments, the active compounds are prepared with carriers that will protect the compound against rapid elimination from the body, such as a controlled release formulation, including but not limited to implants and microencapsulated delivery systems, such as those disclosed in International Publication No. WO 2010/093944, hereby incorporated by reference in its entirety, and specifically with respect to the formulations disclosed therein. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

The compositions and methods of the present disclosure may be utilized to treat a subject in need thereof. In certain embodiments, the subject is a mammal such as a human, or a non-human mammal. When administered to an animal, such as a human, the composition is preferably administered as a pharmaceutical composition comprising, for example, a composition of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In preferred embodiments, when such pharmaceutical compositions are for human administration, particularly for invasive routes of administration (i.e., routes, such as injection or implantation, that circumvent transport or diffusion through an epithelial barrier), e.g., for parenteral administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, powder, syrup, suppository, injection or the like. The composition can also be present in a transdermal delivery system, e.g., a skin patch. The composition can also be present in a solution suitable for topical administration, such as a lotion, cream, ointment or an eye drop.

Certain compounds contained in compositions of the present disclosure may exist in particular geometric or stereoisomeric forms. In addition, polymers of the present disclosure may also be optically active. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

If, for instance, a particular enantiomer of compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compositions may also be formulated for inhalation. In certain embodiments, a composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In some embodiments of the present disclosure, the composition that is suitable for use in the disclosure may be administered orally, topically or parenterally, and in particular topically.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The composition may be mixed under sterile conditions with a pharmaceutically acceptable carrier, and with any preservatives, buffers, or propellants that may be required.

The ointments, pastes, creams and gels may contain, in addition to an antibiotic, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The composition of the disclosure may be formulated with an excipient and component that is common for such oral compositions or food supplements, e.g., especially fatty and/or aqueous components, humectants, thickeners, preserving agents, texturizers, flavor enhancers and/or coating agents, antioxidants and preserving agents. Formulating agents and excipients for oral compositions, and especially for food supplements, are known in this field and will not be the subject of a detailed description herein.

In the case of a composition in accordance with the disclosure for oral administration, the use of an ingestible support is preferred. The ingestible support may be of diverse nature according to the type of composition under consideration. Tablets, gel capsules or lozenges, suspensions, oral supplements in dry form and oral supplements in liquid form are especially suitable for use as food supports.

Formulation of the oral compositions according to the disclosure may be performed via any usual process known to those skilled in the art for producing drinkable solutions, sugar-coated tablets, gel capsules, gels, emulsions, tablets to be swallowed or chewed, wafer capsules, especially soft or hard wafer capsules, granules to be dissolved, syrups, solid or liquid foods, and hydrogels allowing controlled release. Formulation of the oral compositions according to the disclosure may be incorporated into any form of food supplement or enriched food, for example food bars, or compacted or loose powders. The powders may be diluted with water, with soda, with dairy products or soybean derivatives, or may be incorporated into food bars.

In some embodiments, the composition according to the disclosure administered orally may be formulated in the form of sugar-coated tablets, gel capsules, gels, emulsions, tablets, wafer capsules, hydrogels, food bars, compacted or loose powders, liquid suspensions or solutions, confectioneries, fermented milks, fermented cheeses, chewing gum, toothpaste or spray solutions.

An effective amount of the composition may be administered in a single dose per day or in fractional doses over the day, for example two to three times a day. By way of example, the administration of a composition according to the disclosure may be performed at a rate, for example, of 3 times a day or more, generally over a prolonged period of at least a week, 2 weeks, 3 weeks, 4 weeks, or even 4 to 15 weeks, optionally comprising one or more periods of stoppage or being repeated after a period of stoppage.

In certain embodiments, the compound may be administered at a dose between 1 mg and 1,500 mg per day, such as between 5 mg and 1,300 mg per day, such as between 10 mg and 900 mg per day, such as between 20 mg and 600 mg per day, such as between 40 mg and 300 mg per day, such as between 150 mg and 350 mg per day, such as between 40 and 150 mg per day, such as between 25 mg and 150 mg per day, such as between 2.5 mg and 150 mg per day, such as between 20 mg and 80 mg per day, or such as between 1 mg and 30 mg per day. In certain embodiments, the compound may be administered at a dose of 1,300 mg/day, 900 mg/day, 600 mg/day, 350 mg/day, 300 mg/day, 250 mg/day, 200 mg/day, 150 mg/day, 80 mg/day, 75 mg/day, 60 mg/day, 40 mg/day, 30 mg/day, 20 mg/day, 15 mg/day, 10 mg/day, 5 mg/day, or 2.5 mg/day.

This disclosure includes the use of pharmaceutically acceptable salts of compounds of the disclosure in the compositions and methods of the present disclosure. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of a compound. As another example, the salt may comprise less than one inorganic or organic acid molecule per molecule of base, such as two molecules of a compound per molecule of tartaric acid.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form.

Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, 1-aspartic acid, benzenesulfonic acid, benzoic acid, (+)-camphoric acid, (+)-camphor-O-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

As one of skill in the art will appreciate, compositions of the present disclosure, not having adverse effects upon administration to a subject, may be administered daily to the subject.

Preferred embodiments of this disclosure are described herein. Of course, variations, changes, modifications and substitution of equivalents of those preferred embodiments will become apparent to those of ordinary skill in the art upon reading the foregoing description. The inventors expect skilled artisans to employ such variations, changes, modifications and substitution of equivalents as appropriate, and the inventors intend for the disclosure to be practiced otherwise than specifically described herein. Those of skill in the art will readily recognize a variety of non-critical parameters that could be changed, altered or modified to yield essentially similar results. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While each of the elements of the present disclosure is described herein as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

### III. Methods

The present application further provides a method of inhibiting an activity of a HDAC enzyme in a cell sample, a tissue sample, or a subject. In some embodiments, the method is an in vitro method. In some embodiments, the method is an in vivo method. In some embodiments, the method comprises contacting a cell or tissue (e.g., a cell sample or a tissue sample) having an HDAC enzyme with a compound provided herein (e.g. a compound of Formula (I)), or a pharmaceutically acceptable salt thereof. In some embodiments, the method comprises administering to a subject a compound provided herein, or a pharmaceutically acceptable salt thereof. In some embodiments, inhibiting an activity of a HDAC enzyme comprises deregulating the HDAC enzyme.

In some embodiments, the HDAC enzyme is a class lib HDAC enzyme. In some embodiments, the histone deacetylase (HDAC) enzyme is HDAC6.

In certain aspects, the present disclosure provides methods of inhibiting an HDAC enzyme in a subject, comprising administering to the subject a compound or composition as disclosed herein.

As used herein, the term “subject,” refers to any animal, including mammals. Example subjects include, but are not limited to, mice, rats, rabbits, dogs, cats, swine, cattle, sheep, horses, primates, and humans. In some embodiments, the subject is a human. In some embodiments, the method comprises administering to the subject a therapeutically effective amount of a compound provided herein (e.g., a compound of any of Formula (I)), or a pharmaceutically acceptable salt thereof.

The compounds provided herein can be selective HDAC inhibitors. As used, the term “selective” means that the compound binds to or inhibits a particular enzyme (e.g., an isoform of HDAC) with greater affinity or potency, respectively, as compared to at least one other enzyme (e.g., another isoform or all other isoforms of HDAC). In some embodiments, selectivity comprises about 2-fold to about 1000-fold selectivity for a particular isoform as compared to at least one other isoform, for example, about 2-fold to about 1000-fold, about 2-fold to about 500-fold, about 2-fold to about 100-fold, about 2-fold to about 50-fold, about 2-fold to about 20-fold, about 2-fold to about 10-fold, about 10-fold to about 1000-fold, about 10-fold to about 500-fold, about 10-fold to about 100-fold, about 10-fold to about 50-fold, about 10-fold to about 20-fold, about 20-fold to about 1000-fold, about 20-fold to about 500-fold, about 20-fold to about 100-fold, about 20-fold to about 50-fold, about 50-fold to about 1000-fold, about 50-fold to about 500-fold, about 50-fold to about 100-fold, about 100-fold to about 1000-fold, about 100-fold to about 500-fold, or about 500-fold to about 1000-fold.

In some embodiments, the compound provided herein, or a pharmaceutically acceptable salt thereof, selectively inhibits HDAC6 over one or more of HDAC1, HDAC2, HDAC3, HDAC4, HDAC5, HDAC7, HDAC8, HDAC9, HDAC10, and HDAC11.

In some embodiments, the disease is associated with abnormal expression or abnormal activity of a HDAC enzyme in a subject. In some embodiments, the disease to be imaged is associated with abnormal expression or abnormal activity of HDAC6.

The present application further provides a method of treating a disease in a subject in need thereof, the method comprising administering to the subject a therapeutically effective amount of a compound provided herein, or a pharmaceutically acceptable salt thereof. In some embodiments, the disease is associated with abnormal expression or abnormal activity of a HDAC enzyme. In some embodiments, the disease is selected from cancer, a disease of the central nervous system, and an inflammatory autoimmune disease.

In some embodiments, the disease is cancer. In some embodiments, the cancer is selected from breast cancer, prostate cancer, colon cancer, endometrial cancer, brain cancer (e.g., glioblastoma multiforme), bladder cancer, skin cancer, cancer of the uterus, cancer of the ovary, lung cancer, pancreatic cancer, renal cancer, gastric cancer, and hematological cancer. In some embodiments, the cancer comprises a solid tumor. In some embodiments, the cancer is selected from glioma, glioblastoma, non-small cell lung cancer, and hematological cancer. In some embodiments, the cancer is selected from glioma, e.g., glioblastoma; hematological cancer, e.g., leukemia or lymphoma; and non-small cell lung cancer.

In some embodiments, the cancer is a hematological cancer. In some embodiments, the hematological cancer is selected from leukemia and lymphoma. In some embodiments, a hematological cancer is selected from acute myeloblastic leukemia, chronic myeloid leukemia, B cell lymphoma, chronic lymphocytic leukemia (CLL), Non-Hodgkins lymphoma, hairy cell leukemia, Mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma, and germinal center B cell (GCB) diffuse large B cell lymphoma. In some embodiments, the cancer is associated with abnormal expression or abnormal activity of HDAC6.

In some embodiments, the disease to be treated is a disease of the central nervous system. In some such embodiments, the disease is a disease of the central nervous system, such as a disease of the central nervous system selected from mood and mental disorders, e.g., depression, schizophrenia, or bipolar disorder; neurodegenerative diseases, e.g., Huntington's disease, or Alzheimer's disease; drug addiction, e.g., cocaine addiction; and disorders of learning, memory, or cognition. In some embodiments, the disease of the central nervous system is selected from Alzehimer's disease, attention deficit/hyperactivity disorder (ADHD), Bell's Palsy, bipolar disorder, catalepsy, Cerebal Palsy, epilepsy, encephalitis, Huntington's disease, locked-in syndrome, meningitis, migraine, multiple sclerosis (MS), Parkinson's disease, Rett syndrome, schizophrenia, tropical spastic paraparesis, and Tourette's syndrome. In some embodiments, the disease of the central nervous system is selected from Alzheimer's disease, bipolar disorder, depression, Huntington's disease, and schizophrenia. In some embodiments, the disease of the central nervous system comprises a neurodegenerative disease (e.g., amyotrophic lateral sclerosis (ALS), Parkinson's disease, Alzheimer's disease, Huntington's disease, and the like). In some embodiments, the disease of the central nervous system is selected from schizophrenia, bipolar disorder, Alzheimer's disease, and Huntington's disease. In some embodiments, the disease of the central nervous system further comprises depression. In some embodiments, the disease of the central nervous system is depression. In some embodiments, the disease of the central nervous system is associated with abnormal expression or abnormal activity of HDAC6.

In some embodiments, the disease to be treated is an inflammatory autoimmune disease. In some embodiments, the inflammatory autoimmune disease is selected from alopecia areata, autoimmune hemolytic anemia, autoimmune hepatitis, dermatomyositis, diabetes (type 1), juvenile idiopathic arthritis, glomerulonephritis, Graves' disease, Guillain-Barre syndrome, idiopathic thrombocytopenic purpura, myasthenia gravis, myocarditis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus erythematosus, thyroiditis, uveitis, vitiligo, and granulomatosis with polyangiitis (Wegener's granulomatosis). In some embodiments, the inflammatory autoimmune disease is associated with abnormal expression or abnormal activity of HDAC6.

In some embodiments, about 0.1% to about 5% of the compound or salt administered to the subject crosses the blood brain barrier, for example, from about 0.1% to about 4%, from about 0.1% to about 3%, from about 0.1% to about 2%, from about 0.1% to about 1%, from about 0.1% to about 0.75%, from about 0.1% to about 0.5%, from about 0.1% to about 0.25%, from about 0.25% to about 5%, from about 0.25% to about 4%, from about 0.25% to about 3%, from about 0.25% to about 2%, from about 0.25% to about 1%, from about 0.25% to about 0.75%, from about 0.25% to about 0.5%, from about 0.5% to about 5%, from about 0.5% to about 4%, from about 0.5% to about 3%, from about 0.5% to about 2%, from about 0.5% to about 1%, from about 0.5% to about 0.75%, from about 0.75% to about 5%, from about 0.75% to about 4%, from about 0.75% to about 3%, from about 0.75% to about 2%, from about 0.75% to about 1%, from about 1% to about 5%, from about 1% to about 4%, from about 1% to about 3%, from about 1% to about 2%, from about 2% to about 5%, from about 2% to about 4%, from about 2% to about 3%, from about 3% to about 5%, from about 3% to about 4%, or from about 4% to about 5%.

In some embodiments, at least about 5% by weight of the compound or salt administered to the subject crosses the blood-brain barrier, preferably at least about 10% by weight, more preferably at least about 15% by weight, even more preferably at least about 20% by weight of the compound or salt administered to the subject crosses the blood-brain barrier, e.g., to be present in the CNS at therapeutically relevant doses. Selected compounds can be evaluated for CNS penetration by determining plasma and brain levels following i.v., oral, subcutaneous or intraperitoneal administration.

In some embodiments, the compound administering to the subject has a blood:plasma ratio of from about 1:1 to about 100:1, for example, from about 1:1 to about 2:1, from about 1:1 to about 3:1, from about 1:1 to about 4:1, from about 1:1 to about 5:1, from about 1:1 to about 10:1, from about 1:1 to about 15:1, from about 1:1 to about 20:1, from about 1:1 to about 30:1, from about 1:1 to about 1:40, from about 1:1 to about 50:1, from about 1:1 to about 60:1, from about 1:1 to about 70:1, from about 1:1 to about 80:1, from about 1:1 to about 90:1, from about 1:1 to about 100:1, from about 1:1 to about 3:2, or from about 1:1 to about 4:3. In some embodiments, the blood:plasma ratio is from about 1:100 to about 1:1, for example, from about 1:100 to about 1:1, from about 1:100 to about 1:2, from about 1:100 to about 1:3, from about 1:100 to about 1:4, from about 1:100 to about 1:5, from about 1:100 to about 1:10, from about 1:100 to about 1:15, from about 1:100 to about 1:20, from about 1:100 to about 1:30, from about 1:100 to about 1:40, from about 1:100 to about 1:50, from about 1:100 to about 1:60, from about 1:100 to about 1:70, from about 1:100 to about 1:80, from about 1:100 to about 1:90, at least about 1:100, from about 1:100 to about 2:3, from about 1:100 to about 2:5, from about 1:100 to about 3:4, from about 1:100 to about 3:5, or from about 1:100 to about 4:5. In some embodiments, the compound administered has a brain plasma ratio of from about 1:1 to about 50:1.

As used herein, the phrase “therapeutically effective amount” refers to the amount of active compound or pharmaceutical agent that elicits the intended biological or medicinal response in a tissue, system, animal, individual or human.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

One or more additional therapeutic agents such as, for example, chemotherapeutic agents, anti-inflammatory agents, steroids, immunosuppressants, therapeutic antibodies, and/or anesthetics, can be used in combination with the compounds and salts provided herein for treatment of HDAC associated diseases, disorders, or conditions.

Example chemotherapeutic agents include proteosome inhibitors (e.g., bortezomib), thalidomide, revlimid, and DNA-damaging agents such as melphalan, doxorubicin, cyclophosphamide, vincristine, etoposide, carmustine, and the like.

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example steroids include, but are not limited to, corticosteroids such as cortisone, dexamethasone, hydrocortisone, methylprednisolone, prednisolone, and prednisone.

Example immunosuppressants include, but are not limited to, azathioprine, chlorambucil, cyclophosphamide, cyclosporine, daclizumab, infliximab, methotrexate, and tacrolimus.

Example anesthetics include, but are not limited, to local anesthetics (e.g., lidocaine, procain, ropivacaine) and general anesthetics (e.g., desflurane, enflurane, halothane, isoflurane, methoxyflurane, nitrous oxide, sevoflurane, amobarbital, methohexital, thiamylal, thiopental, diazepam, lorazepam, midazolam, etomidate, ketamine, propofol, alfentanil, fentanyl, remifentanil, buprenorphine, butorphanol, hydromorphone levorphanol, meperidine, methadone, morphine, nalbuphine, oxymorphone, pentazocine).

In some embodiments, the additional therapeutic agent is administered simultaneously with a compound or salt provided herein. In some embodiments, the additional therapeutic agent is administered after administration of the compound or salt provided herein. In some embodiments, the additional therapeutic agent is administered prior to administration of the compound or salt provided herein. In some embodiments, the compound or salt provided herein is administered during a surgical procedure. In some embodiments, the compound or salt provided herein is administered in combination with an additional therapeutic agent during a surgical procedure.

### IV. Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C. A. (1985); “Carboranes, 3rd ed.”, R. N. Grimes, Academic Press, New York, (2016); and “Boron Hydrides”, W. N. Lipscomb, Benjamin, New York, (1963).

All of the above, and any other publications, patents and published patent applications referred to in this application are specifically incorporated by reference herein. In case of conflict, the present specification, including its specific definitions, will control.

The terms “a,” “an,” “the” and similar referents used in the context of describing the present disclosure are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the disclosure otherwise claimed. No language in the present specification should be construed as indicating any unclaimed element is essential to the practice of the disclosure.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The term “acetal” is art-recognized and may be represented by the general formula

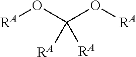

wherein each R^{A} independently represents hydrogen or a hydrocarbyl, such as alkyl, or any occurrence of R^{A} taken together with another and the intervening atom(s) complete a carbocycle or heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group. An alkyl group with two open valences is sometimes referred to as an alkylene group, such as methylene, ethylene, propylene and the like.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. Co alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively. As applied to heteroalkyls, “C_{x-y}” indicates that the group contains from x to y carbons and heteroatoms in the chain. As applied to carbocyclic structures, such as aryl and cycloalkyl groups, “C_{x-y}” indicates that the ring comprises x to y carbon atoms. As applied to heterocyclic structures, such as heteroaryl and heterocyclyl groups, “C_{x-y}” indicates that the ring contains from x to y carbons and heteroatoms. As applied to groups, such as aralkyl and heterocyclylalkyl groups, that have both ring and chain components, “C_{x-y}” indicates that the ring and the chain together contain from x to y carbon atoms and, as appropriate heteroatoms.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

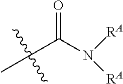

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

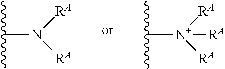

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “boron” as used herein with respect to a substituent on an organic compound, is art-recognized and refers to a group —B(R^{A})₂, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “boronic ester” or “boronate ester” as used herein is art-recognized and refers to a group —B(OR^{A})₂, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “carbamate” is art-recognized and refers to a group

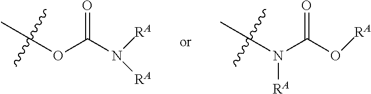

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “diazo”, as used herein, refers to a group represented by the formula ═N═N.

The term “disulfide” is art-recognized and refers to a group —S—S—R^{A}, wherein R^{A} represents a hydrocarbyl group.

The term “enol ester”, as used herein, refers to a group —C(O)O—C(R^{A})═C(R^{A})₂ wherein R^{A} represents a hydrocarbyl group.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent. In analogy with alkyl groups, heteroalkyl groups with two open valences are sometimes referred to as heteroalkylene groups. Preferably, the heteroatoms in heteroalkyl groups are selected from O and N.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, “mitigating” means reducing the negative effects caused by exposure to ionizing radiation, relative to a cell, organ, tissue, or organism exposed to the same level of radiation for the same amount of time, but untreated.

As used herein, a “therapeutically effective amount” is an amount sufficient to mitigate the effects of the ionizing radiation.

The term “orthoester” as used herein is art-recognized and refers to a group —C(OR^{A})₃, wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

The term “phosphoester”, as used herein, refers to a group —P(O₂)OH.

The term “phosphodiester”, as used herein, refers to a group —P(O₂)OR^{A} wherein R^{A} represents a hydrocarbyl group.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7. When a polycyclic substituent is attached through an aryl or heteroaryl ring, that substituent may be referred to herein as an aryl or heteroaryl group, while if the polycyclic substituent is attached through a cycloalkyl or heterocyclyl group, that substituent may be referred to herein as a cycloalkyl or heterocyclyl group. By way of example, a 1,2,3,4-tetrahydronaphthalen-1-yl group would be a cycloalkyl group, while a 1,2,3,4-tetrahydronaphthalen-5-yl group would be an aryl group.

The term “selenide”, as used herein, is equivalent to an ether, wherein the oxygen is replaced with a selenium.

The term “selenoxide” is art-recognized and refers to the group —Se(O)—R^{A}, wherein R^{A} represents a hydrocarbyl.

The term “siloxane” is art-recognized and refers to a group with an Si—O—Si linkage, such as the group —Si(R^{A})₂—O—Si—(R^{A})₃, wherein each R^{A} independently represents hydrogen or hydrocarbyl, such as alkyl, or both R^{A} taken together with the intervening atom(s) complete a heterocycle having from 4 to 8 atoms in the ring structure.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

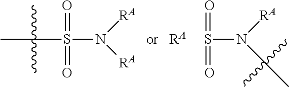

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

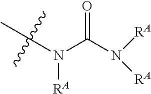

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula (I). Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula (I) are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula (I) for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula (I) or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixtures and separate individual isomers.

Some of the compounds may also exist in tautomeric forms. Such forms, although not explicitly indicated in the formulae described herein, are intended to be included within the scope of the present disclosure.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting, or transitory, that can be associated with the administration of the pharmaceutical composition.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Prodrug” or “pharmaceutically acceptable prodrug” refers to a compound that is metabolized, for example hydrolyzed or oxidized, in the host after administration to form the compound of the present disclosure (e.g., compounds of formula (I)). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula (I). The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” is art-recognized and includes administration to the host of one or more of the subject compositions, e.g., to diminish, ameliorate, or stabilize the existing unwanted condition or side effects thereof.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The disclosure now being generally described, it will be more readily understood by reference to the following examples which are included merely for purposes of illustration of certain aspects and embodiments of the present disclosure, and are not intended to limit the disclosure.

In one aspect, the present disclosure relates to a process for preparing a compound of Formula (I), e.g., comprising preparing a compound of Formula (II) by reacting a compound of Formula (III) with a compound of Formula (IV) under reductive alkylation conditions:

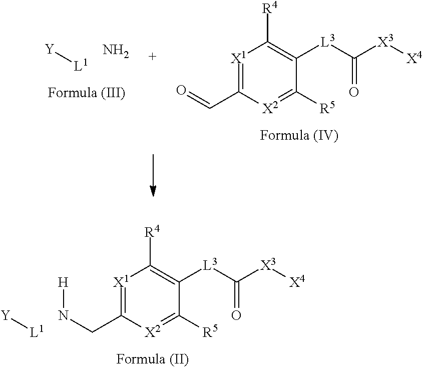

The process for preparing a compound of Formula (II) under reductive alkylation conditions comprising reacting an aldehyde group or keto group or hemiacetal group with at least one amino group of the carborane. The synthesis of exemplary compounds disclosed herein is shown in the examples included herein.

In some embodiments, the process can include introducing at least one aldehyde group or keto group or hemiacetal group being functionally modified, such as a compound of Formula IV, to react with at least one amino group of the carborane by reductive alkylation.

In certain embodiments, the reductive alkylation can be carried out in an aqueous medium in the presence of a reducing agent, such as NaCNBH₃ or sodium triacetoxyborohydride.

In some embodiments, the reductive alkylation can be carried out at a pH of about 7.5, preferably about 7 or less and can be carried out at a temperature of about −5 to about 100° C., preferably at about 0 to about 25° C.

### Example 1. General Considerations

1,7-dicarbo-closo-decaborane (meta-C₂B₁₀H₁₂, KatChem) was sublimed prior to use. Bromine Sigma-Aldrich, reagent grade) and aluminium (III) chloride (Sigma-Aldrich, ReagentPlus, 99%) were used as received. Anhydrous K₃PO₄ (Sigma-Aldrich, anhydrous, free-flowing, Redi-Dri, reagent grade, >98%) was stored in a N₂ filled glovebox and used as received. N,N-diisopropylethylamine (Sigma-Aldrich, ReagentPlus, >99%) was dried with 4 Å molecular sieves and sparged with N₂ before use. 1,4-dioxane (Sigma-Aldrich, anhydrous, 99.8%) was stored over 4 Å molecular sieves prior to use, anhydrous toluene (Sigma-Aldrich, 99%), dry dichloromethane and THE were obtained from a Grubbs column with an activated alumina column, THE was stored over 4 Å molecular sieves inside a N₂ filled glovebox.¹ Adamantane carbonitrile (Alfa Aesar) was used as received. 2-Dicyclohexylphosphino-2′,6′-dimethoxybiphenyl (SPhos, Sigma-Aldrich, 97%), 2-Dicyclohexylphosphino-2′,4′,6′-triisopropylbiphenyl (XPhos, Matrix Scientific, 95%) were used as received. XPhos-Pd-G3 and SPhos-Pd-G3 precatalysts were prepared according to ref 2.

Silica gel (Fisher, Grade 60, 230-400 Mesh), Celite (Fisher, 545 filter aid, not acid washed, powder) were used as received. Deuterated chloroform (CDCl₃) and methanol (CD₃OD) (Cambridge Isotope Laboratories) were used as received.

All cross-coupling reactions were performed in an oven dried 16 mL reaction tube (Fisher, 16 mm diameter, borosilicate glass) fitted with a PTFE lined magnetic stir bar and PTFE lined septum cap. Glass-backed Silica Gel 60 GLA thin-layer chromatography (TLC) plates (Silicycle) were used as received. TLC samples for carborane-containing compounds were stained with 1 wt. % PdCl₂ in 6 M HCl and were developed with high heat using a heat gun to produce black stains indicative of carborane containing molecules.

Characterization

¹H, ¹³C{¹H}, ¹¹B NMR spectra were recorded on AV400 spectrometers in ambient conditions unless stated otherwise. Bruker Topspin V3.2 and MestReNova 11.0 software was used to process the FID data to visualize the spectra. ¹H and ¹³C{¹H} NMR spectra were referenced to residual solvent resonances in deuterated solvents (CDCl₃: ¹H, 7.26 ppm; ¹³C, 77.16 ppm, Note: due to high humidity H₂O resonances are often present) and are reported relative to tetramethylsilane (d=0 ppm). ¹¹B and ¹¹B{¹H} NMR spectra were referenced to Et₂O.BF₃ in a sealed capillary (d=0 ppm).

Gas Chromatography Mass Spectrometry (GC-MS) data were collected on an Agilent 6890-5975 GC-MS equipped with an Agilent J&W HP-5 column with He carrier gas.

Chinese hamster ovary (CHO) cells were purchased from ATCC and cultured in Corning™ Cellgro™ F12K Medium (cat #MT10025CV) supplemented with 10% fetal bovine serum (Corning™, lot #35016109) and 1% penicillin-streptomycin (Life Technologies, cat #15070063). Cells were washed with PBS, or PBS supplemented with 1% fetal bovine serum (FACS buffer). Cells were incubated at 37° C., 5% CO₂, during treatments and throughout culturing, in HERACell 150i CO₂ incubators. Cells were pelleted through use of Sorvall ST 40R centrifuge. All cell work was performed in 1300 Series A2 biosafety cabinets.

Primary neural progenitor cells (NPCs) were a gift from Dr. Harley Kornblum (University of California Los Angeles). The cells were collected from embryonic day 14 (E14) mouse brain according to procedures approved by the UCLA Chancellor's Committee for Animal Research. The following supplements were added to the Neurobasal media: 1:50 B27, 1:100 Pen-Strep, 2 mM 1-glutamine (all from GIBCO/BRL), 2 μg/ml heparin (Sigma), and 20 ng/ml FGF2 (PeproTech). Cells were incubated at 37° C. in the presence of 5% CO₂. Additional FGF2 was added every third day, and cultures were passaged every 7 days. During each passage, spheres were enzymatically and mechanically dissociated with Accumax (Sigma).

Statistical Analysis. For assessment of the statistical significance of differences, one-tailed Student's t-test assuming unequal sample variance was employed. Results were considered significantly different if p<0.05.

### Example 2A. Synthesis of Carboranostat

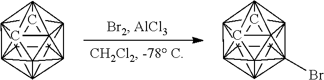

Bromo carborane was prepared as described previously (reference: Dziedzic, R. M., Saleh, L. M. A., Axtell, J. C., Martin, J. L., Stevens, S. L., Royappa, A. T., Rheingold, A. L., and Spokoyny, A. M. J. Am. Chem. Soc., 2016, 138, 9081-9084.)

### 9-bromo-m-carborane

Meta-C₂B₁₀H₁₂ (1.44 g, 10 mmol) was added to an oven dried Schlenk flask. The flask was evacuated and backfilled with N₂ three times. Dry dichloromethane (50 mL) and Br₂ (1.60 g, 10 mmol) were added to the reaction vessel and then cooled to −78° C. in a dry ice/acetone bath. AlCl₃ (0.130 g, 10 mol %) was added to the flask containing the rapidly stirring carborane/Br₂ solution at −78° C. under an N₂ flow. The reaction vessel was sealed with a glass stopper and stirred for 20 minutes at −78° C., after which the reaction mixture was allowed to warm to room temperature and stirred for 2 hours. The reaction solution became colorless and completion of the reaction was determined by GC-MS. The contents of the flask were subsequently slowly quenched with distilled H₂O (20 mL) yielding a cloudy suspension. The cloudy organic layer was separated from the aqueous layer and the aqueous layer was extracted with dichloromethane (2×10 mL). The organic portions were then combined and dried over MgSO₄ resulting in a clear colorless solution. The solution was then filtered through a silica pad on a fritted funnel and the filtrate removed under reduced pressure to yield a white solid, which was further dried in vacuo to produce the title compound as a white solid (94%).

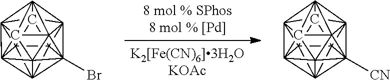

Cyano carborane was prepared as previously described (reference: Dziedzic, R. M., Saleh, L. M. A., Axtell, J. C., Martin, J. L., Stevens, S. L., Royappa, A. T., Rheingold, A. L., and Spokoyny, A. M. J. Am. Chem. Soc., 2016, 138, 9081-9084.) with slight modifications.

### 9-cyano-m-carborane

In an oven dried Schlenk flask, 9-bromo-m-carborane (9-Br-1,7-dicarba-closo-dodecaborane) (1.338 g, 6 mmol, 1 eq.), SPhos (246.3 mg, 0.48 mmol, 8.3 mol %), SPhos precat. (386.2 mg, 0.48 mmol, 8.3 mol %), and finely ground-using ball milling K₄[Fe(CN)₆].3H₂O (1.774 g, 4.2 mmol, 0.7 eq.) were added. The vessel was purged with nitrogen five times. Deoxygenated 1,4-dioxane (6 mL) and deoxygenated 0.1 M KOAc aqueous solution (6 mL) were sequentially added to the vessel using a syringe. The reaction was heated at 100° C. and stirred vigorously for 48 hours. When conversion was confirmed via GC-MS, 30 mL of methylene chloride was added to the mixture. The mixture was then washed with three portions of DI water (20 mL each). The organic layer was separated and dried over MgSO₄, then passed through Celite. The yellow solution was dried and purified first via flash chromatography (9:1 hexanes:DCM for elution of remaining bromo-carborane starting material, then 2:1 hexanes:DCM mixture for elution of product) through a silica column, using PdCl₂ in HCl stain for visualization. The fractions containing the pure product were combined and dried in vacu. The remaining organic impurities were removed by sublimation at 100° C. for 3 hours affording the product as a white powder (63% yield).

¹H NMR (CDCl₃, 400 MHz): δ 3.06 (s, 2H, C1,C7), 1.60-3.43 (br m, 9H, B2-6, B8, B10-12) ppm.

¹¹B{¹H} NMR (CDCl₃, 400 MHz): δ −17.53 (s, 1B), −15.52 (d, 2B), −12.90 (d, 4B), −9.60 (d, 1B), −5.88 (d, 2B) ppm.

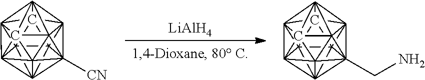

### 9-aminomethyl-m-carborane

A reaction tube was charged with cyano carborane (40 mg, 0.236 mmol, 1 eq.) and LiAlH₄ (36 mg, 0.945 mmol, 4 eq.) and a stir bar. The tube was sealed and purged with nitrogen 5 times. Next, 4 mL of dry 1,4-dioxane was added to the tube and the mixture was heated at 100° C. for 3 hours. After cooling the reaction mixture to room temperature, the reaction tube was placed in an ice bath and in order, 40 μL of water, 40 μL of 15% NaOH and 120 μL of water was slowly and carefully added dropwise to the reaction mixture. The mixture was shaken and stirred until a white powdery precipitate is formed, which is then filtered off. The precipitate is washed with diethyl ether. The solution is then dried to afford product as a white solid (77% yield).

¹¹B NMR (CDCl₃, 400 MHz): δ −20.31 (d, 1B), −17.78 (d, 1B), −13.86 (q, 4B), −10.51 (d, 1B), −6.91 (d, 2B), 1.01 (s, 1B) ppm.

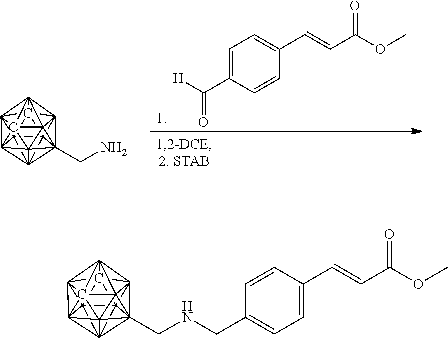

### methyl (E)-3-(4-(((((3r,5r,7r)-m-carboran-9-yl)methyl)amino)methyl)phenyl)acrylate

9-aminomethyl-m-carborane (300 mg, 1.73 mmol, 1.4 eq.), methyl (E)-3-(4-formylphenyl)acrylate (234 mg, 1.23 mmol, 1 eq.), and 50 mg Na₂SO₄ were added to an oven-dried Schlenk flask with a stir bar. The flask was purged with nitrogen 5 times. 10 mL dry 1,2-dichloroethane was added via syringe. The reaction was stirred for 20 hours at room temperature until ¹H NMR spectroscopy revealed complete conversion of the aldehyde to the imine intermediate. Then the reaction mixture was cooled to 0° C. and sodium triacetoxyborohydride (612 mg, 2.89 mmol, 2 eq.) was added to the solution under a positive flow of nitrogen. After 1 hour, the solution was warmed up to room temperature and stirred for 3 hours as monitored by the disappearance of the imine via ¹H NMR. Then the solution was washed with 20 mL NaHCO₃ saturated solution twice, and once with 10 mL of distilled water. The organic layer was dried over Na₂SO₄. The mixture was filtered through Celite on a glass frit and dried in vacuo. The resulting yellow oil was recrystallized in methanol.

¹H NMR (CDCl₃, 400 MHz): δ 7.63 (d, 1H, J=15.94 Hz), 7.52 (d, 2H, J=7.78 Hz), 7.46 (d, 2H, J=7.78 Hz), 6.37 (d, 1H, J=15.97 Hz), 4.04 (s, 2H), 2.95 (br s, 2H), 2.46 (s, 2H) ppm.

¹¹B{¹H} NMR (CDCl₃, 400 MHz): δ −0.38 (1B), −6.63 (2B), −10.30 (1B), −13.94 (4B), −17.6-19.87 (2B) ppm.

MS: Calculated m/z (M+H)⁺: 348.2966; Observed: 348.29846.

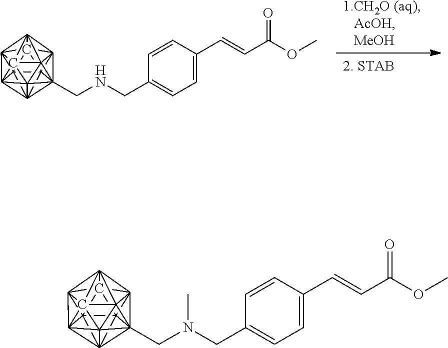

### methyl (E)-3-(4-(((((3r,5r,7r)-carboran-9-yl)methyl)(methyl)amino)methyl)phenyl)acrylate

To a solution of methyl (E)-3-(4-(((((3r,5r,7r)-m-carboran-9-yl)methyl)amino)methyl)phenyl)acrylate (100 mg, 0.576 mmol) in 6 mL methanol, formaldehyde (37% aqueous, 0.4 mL) was added followed by 20 μL acetic acid. The solution was stirred at room temperature for 2 hours. The reaction mixture was cooled to 0° C. and sodium triacetoxyborohydride (305 mg, 2.88 mmol, 5 eq.) was added. After 1 hour, the solution was warmed up to room temperature and stirred for 3 hours. A white precipitate was formed that was filtered off. The solution was then dried and the residue was dissolved in 10 mL methylene chloride and washed twice with 20 mL portions of saturated NaHCO₃. The organic layer was dried over Na₂SO₄ and the mixture was filtered through Celite on a glass frit and dried in vacuo providing the product as a mixture as a yellow oil.

¹H NMR (CDCl₃, 400 MHz): δ 7.69 (d, 1H, J=15.97 Hz), 7.46 (d, 2H, J=7.78 Hz), 7.39 (d, 2H, J=7.78 Hz), 6.41 (d, 1H, J=15.97 Hz), 3.79 (s, 3H), 3.52 (s, 2H), 2.90 (br s, 2H), 2.23 (s, 3H) ppm.

¹¹B{¹H} NMR (CDCl₃, 400 MHz): δ −0.75 (1B), −6.48 (2B), −10.02 (1B), −13.24-14.00 (4B), −17.65-19.78 (2B) ppm.

MS: Calculated m/z (M+H)⁺: 362.3009; Observed: 362.31509.

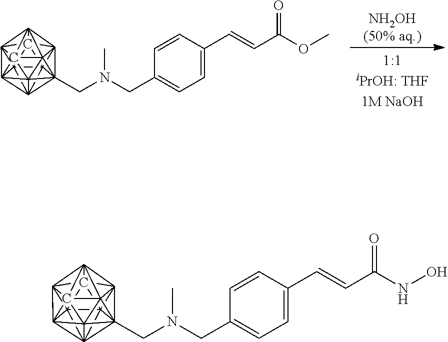

### (E)-3-(4-(((((3r,5r,7r)-adamantan-1-yl)methyl)(methyl)amino)methyl)phenyl)-N-hydroxyacrylamide (Carboranostat)

methyl (E)-3-(4-(((((3r,5r,7r)-carboran-9-yl)methyl)(methyl)amino)methyl)phenyl)acrylate (63.1 mg, 0.175 mmol, 1 eq.) was dissolved in 1 mL of 1:1 MeOH: PrOH mixture. The solution was cooled to 0° C. Hydroxylamine (50% aqueous solution, 374 μL, 6.11 mmol, 35 eq.) and 1 M NaOH (244 μL, 1.4 eq.) were added to the mixture, yielding a clear light yellow solution. The solution was stirred at 0° C. for 2.5 hours and then at room temperature for 2 hours. Next, the organic solvents were removed in vacuo, leaving a cloudy aqueous solution. Upon treatment with 1 M HCl, a white solid precipitate forms. Addition of HCl was continued until the solution was neutralized. The solid was filtered and washed with small portions of water 3 times. The solid was then lyophilized producing 4C as white powder.

MS: Calculated m/z (M+H)⁺: 363.2961; Observed: 363.30917.

### Example 2B. Synthesis of Martinostat

Synthesis of Martinostat was adapted from previously published protocols: Schroeder, F. A.; Wang, C.; Van de Bittner, G. C.; Neelamegam, R.; Takakura, W. R.; Karunakaran, A.; Wey, H. Y.; Reis, S. A.; Gale, J.; Zhang, Y. L.; Holson, E. B.; Haggarty, S. J.; Hooker, J. M. PET imaging demonstrates histone deacetylase target engagement and clarifies brain penetrance of known and novel small molecule inhibitors in rat. ACS Chem. Neurosci. 2014, 5, 1055-1062.

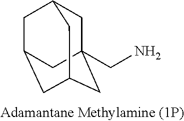 [SMILES: NCC12CC3CC(CC(C3)C1)C2]

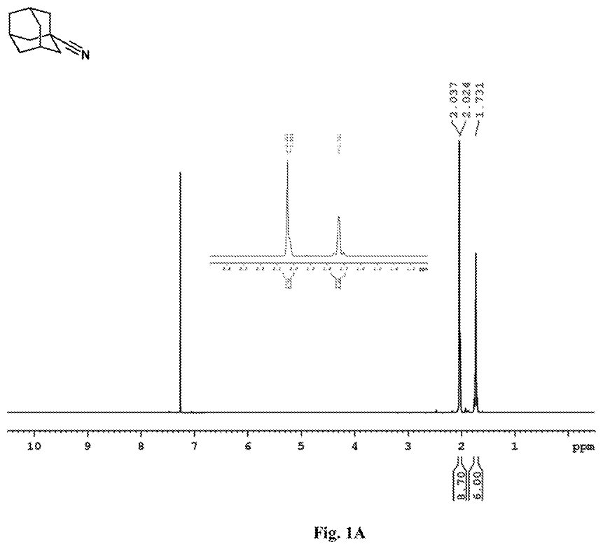

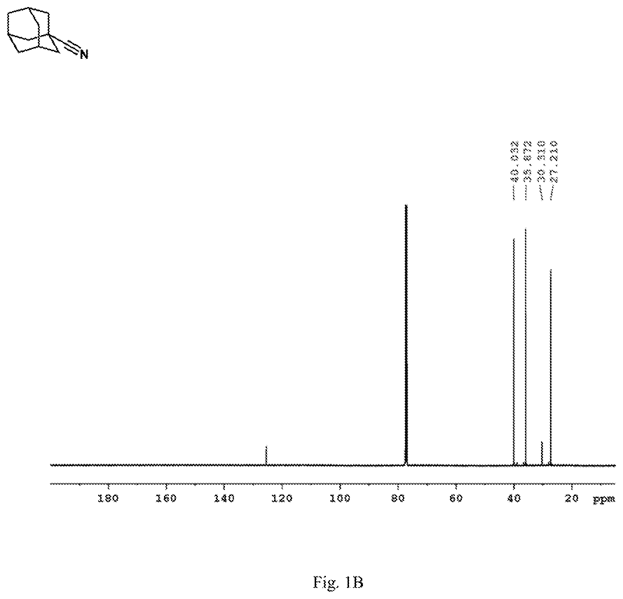

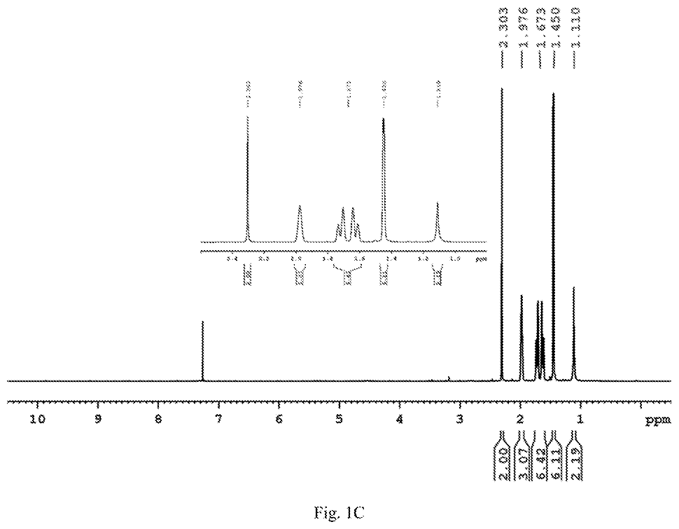

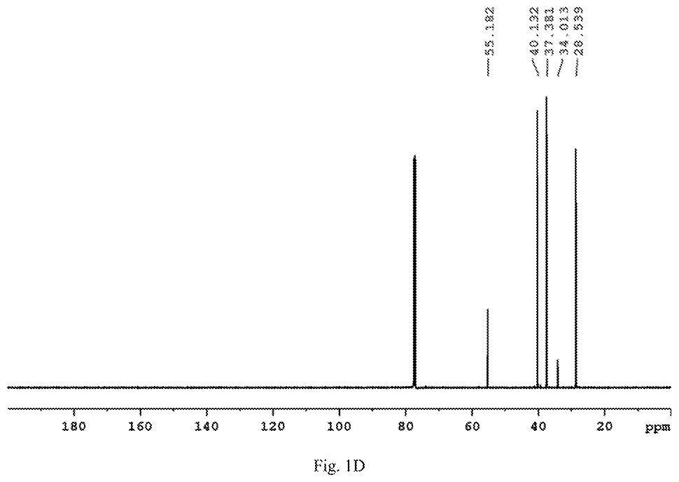

To a solution of LiAlH₄ (1.88 g, 49.6 mmol, 4 eq) in 80 mL dry diethyl ether, adamantane carbonitrile (Alfa Aesar) (2.00 g, 12.4 mmol, 1 eq) dissolved in 30 mL dry diethyl ether was added at 0° C. After 30 minutes the reaction was warmed up to room temperature and left to stir overnight under a positive flow of nitrogen. The reaction mixture was then exposed to air, cooled to 0° C., and 1.88 mL H₂O, 1.88 mL 15% NaOH, and 5.64 mL H₂O were added stepwise and very carefully to the reaction mixture. Stirring is continued until a white precipitate is formed. The mixture was then filtered through a glass frit and washed with 30 mL diethyl ether. The filtrate was dried in air providing 1P as a white solid (1.89 g, 92%). NMR spectra of adamantane carbonitrile are shown in FIGS. 1A (1H NMR) and 1B (13C NMR). NMR spectra of adamantane methylamine are shown in FIGS. 1C (1H NMR) and 1D (13C NMR).

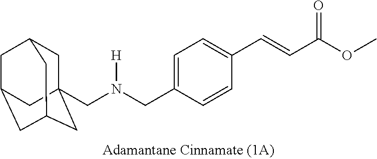 [SMILES: COC(=O)/C=C/c1ccc(CNCC23CC4CC(CC(C4)C2)C3)cc1]

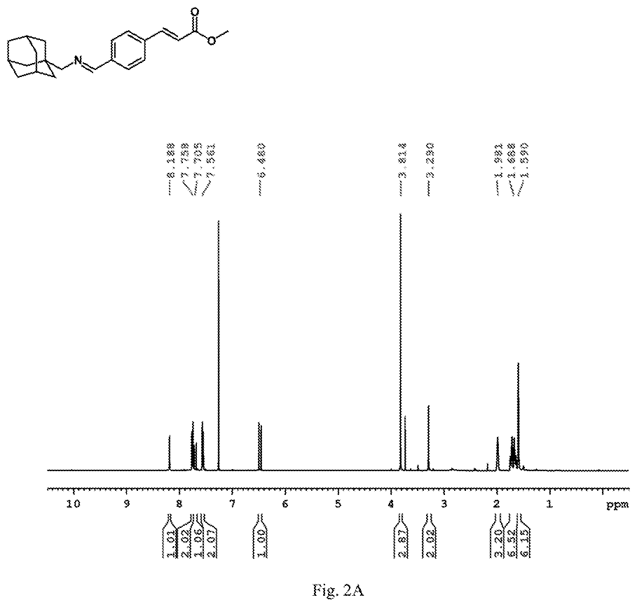

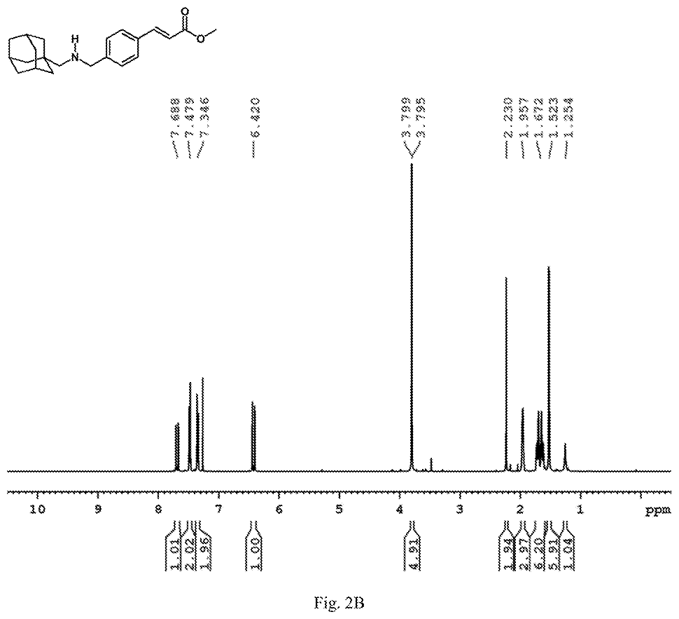

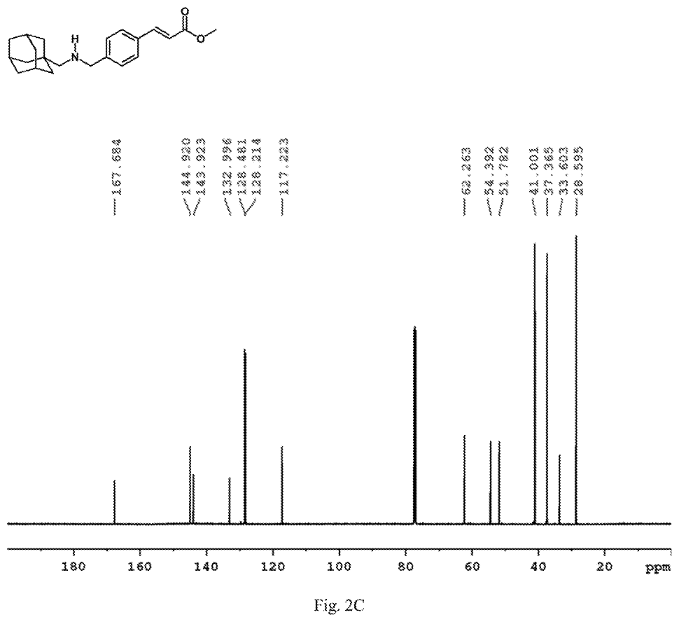

To a mixture of 1P (1.50 g, 9.08 mmol, 1.1 eq) and 1 (1.57 g, 8.25 mmol, 1 eq) in a round bottom flask, 50 mL DCE was added and the solution was stirred overnight at room temperature. Upon confirmation of imine formation via ¹H NMR, the solution was cooled to 0° C. and sodium triacetoxyborohydride (3.85 g, 18.15 mmol, 2 eq) was added. After 45 minutes, the solution was warmed up to room temperature and stirred overnight. The reaction was quenched by addition of 100 mL of 0.5M NaHCO₃. The organic layer was separated and treated with another 100 mL of 0.5M NaHCO₃. Next, the organic layer was washed with 50 mL of DI water and dried over MgSO₄. The mixture was filtered through Celite on a glass frit and dried in vacuo providing a yellow oil. The oil was dissolved in 5 mL MeOH and cooled to −15° C. for at least 15 minutes. The white precipitate was filtered off and washed with cold MeOH and cold Ether to obtain the product 1A (2.12 g, 76%). FIG. 2A shows a 1H NMR spectrum of adamantane cinnamate imine intermediate, FIG. 2B shows a 1H NMR spectrum of adamantane cinnamate, and FIG. 2C shows a 13C NMR spectrum of adamantane cinnamate.

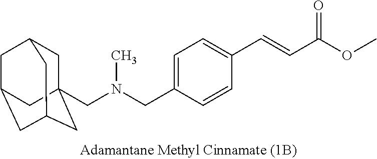 [SMILES: COC(=O)/C=C/c1ccc(CN(C)CC23CC4CC(CC(C4)C2)C3)cc1]

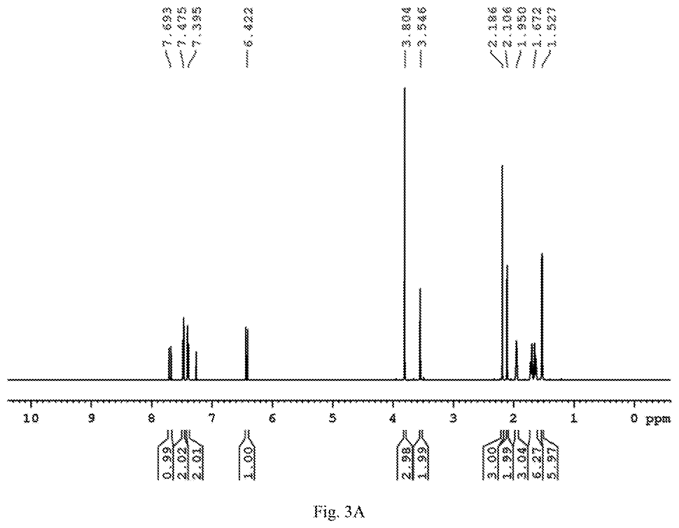

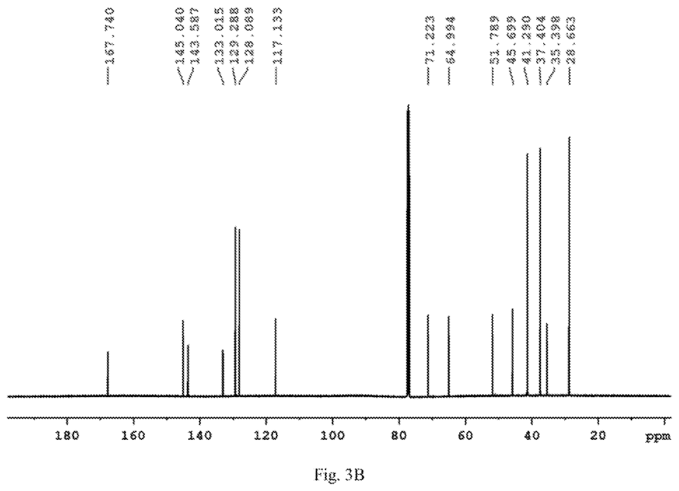

To a solution of 1A (1.500 g, 4.42 mmol) in 90 mL methanol, formaldehyde (37% aqueous, 6 mL) was added followed by 0.30 mL acetic acid. The solution was stirred at room temperature for 2 hours. The reaction mixture was cooled to 0° C. and sodium triacetoxyborohydride (4.686 g, 22.11 mmol) was added. After 45 minutes, the solution was warmed up to room temperature and stirred overnight. The solvent was then removed and the residue was dissolved in 100 mL methylene chloride and washed twice with 30 mL portions of saturated NaHCO₃. The organic layer was washed with 50 mL DI water and then dried over MgSO₄. The mixture was filtered through Celite on a glass frit and dried in vacuo providing a yellow oil. The oil was dissolved in minimal amount of 2:1 MeOH:Et₂O solution and cooled at −15° C. The product, 1B, precipitated out of solution in form of white crystals (1.2773 g, 82%). FIGS. 3A-3B show 1H NMR (FIG. 3A) and 13C NMR (FIG. 3B) spectra of the product. MS: Calculated m/z (M+H)⁺: 354.2433; Observed: 354.2484.

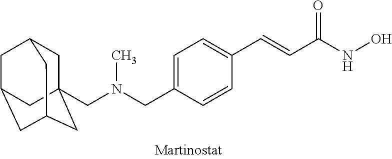 [SMILES: CN(Cc1ccc(/C=C/C(=O)NO)cc1)CC12CC3CC(CC(C3)C1)C2]

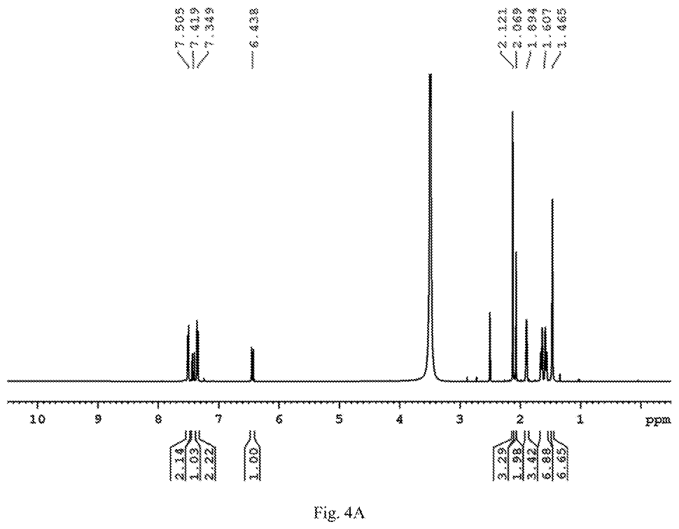

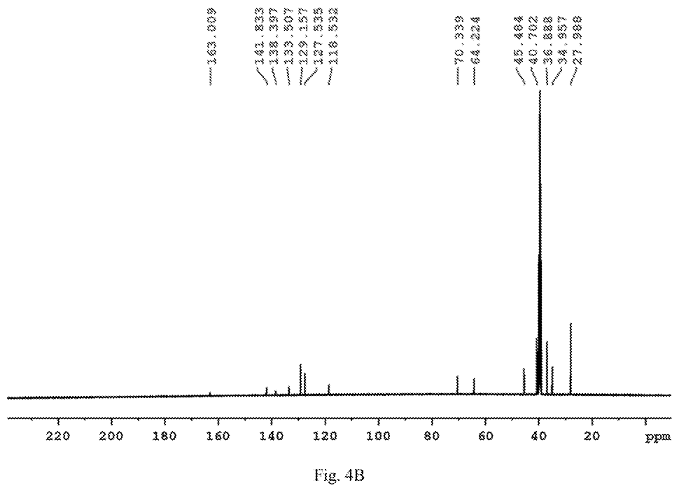

1B (338 mg, 0.956 mmol, 1 eq) was dissolved in 4 mL of 1:1 ⁱPrOH:THF mixture. The solution was cooled to 0° C. Hydroxylamine (50% aqueous solution, 2.05 mL, 33.46 mmol, 35 eq) and 1M NaOH (1.34 mL, 1.4 eq) were added to the mixture, yielding a clear yellow solution. The solution was stirred at 0° C. for 2.5 hours and then at room temperature for 4 hours. Next, the organic solvents were removed in vacuo, leaving a cloudy aqueous solution with pH around 12-13 (as measured by pH paper). Upon treatment with 1M HCl a white solid precipitate forms. Addition of HCl was continued until the solution turned neutral (pH 7 as indicated by pH paper). The supernatant was removed and the solid was washed with small portions of water 5 times. The solid was then lyophilized producing 1C as white powder (323 mg, 95%). FIGS. 4A-4B show 1H NMR (FIG. 4A) and 13C NMR (FIG. 4B) spectra of the product. MS: Calculated m/z (M+H)⁺: 355.2386; Observed: 355.2431.

### Example 2C: Synthesis of Azinostat

Azinostat is derived from an amino carborane, whose synthesis is previously published. The following steps to the final compound are the same as for Carboranostat.

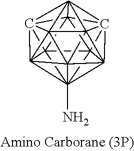

Synthesis is from reference: Dziedzic, R. M., Saleh, L. M. A., Axtell, J. C., Martin, J. L., Stevens, S. L., Royappa, A. T., Rheingold, A. L., and Spokoyny, A. M. J. Am. Chem. Soc., 2016, 138, 9081-9084.

A 25 mL Schlenk flask was charged with SPhos (41.0 mg, 0.05 eq.), SPhos-Pd-G3 precatalyst (79.6 mg, 0.05 eq.), and Bromo Carborane (446 mg, 2 mmol, 1 eq.). The flask was evacuated and backfilled with N₂ four times and transferred to a N₂ filled glovebox, where K^{t}BuO (672 mg, 3 eq.) was added to the flask. The flask was resealed and transferred out of the glovebox. Next, 12 mL of 0.5M NH₃ in 1,4-dioxane was injected in the reaction vessel by a syringe. The mixture was heated for 2 hours at 80° C. The reaction mixture was then filtered through alumina using ethylacetate and the solvent was removed under reduced pressure to yield a yellow oil. The oil was sublimed at 80° C. for 3 hours to afford the final product as white crystals (50%).

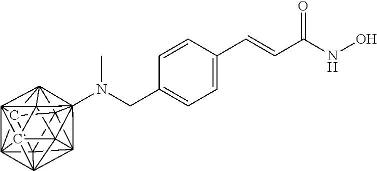

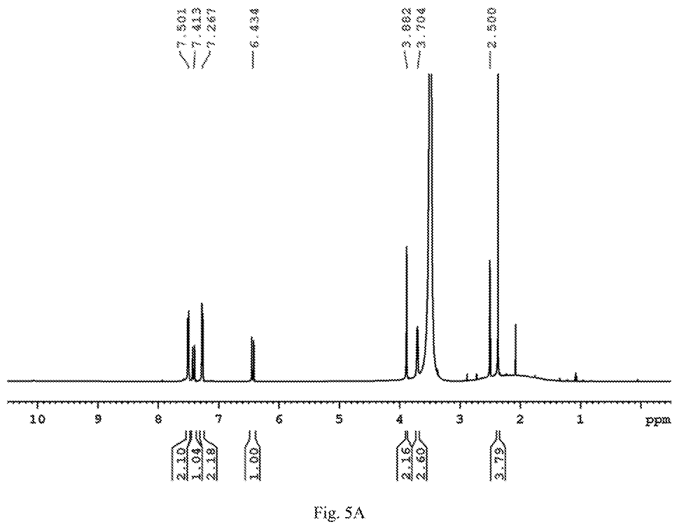

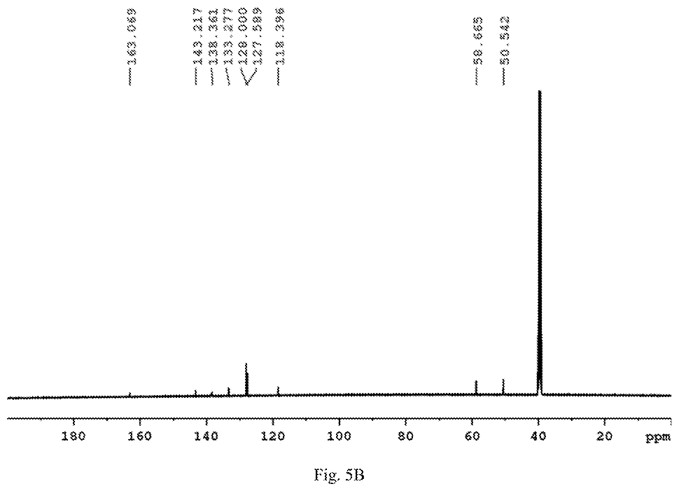

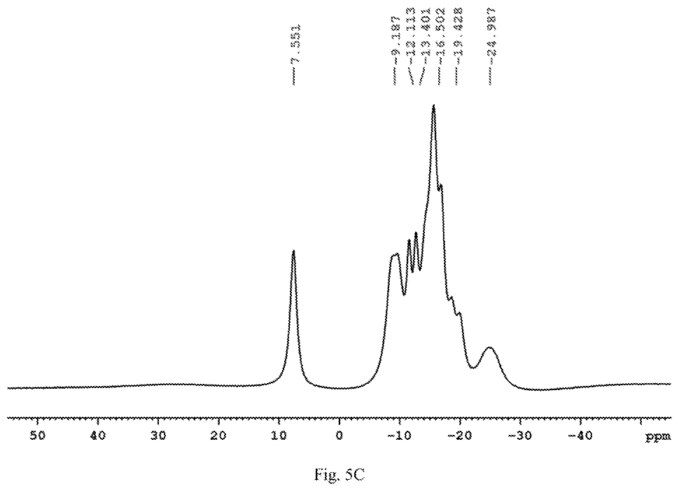

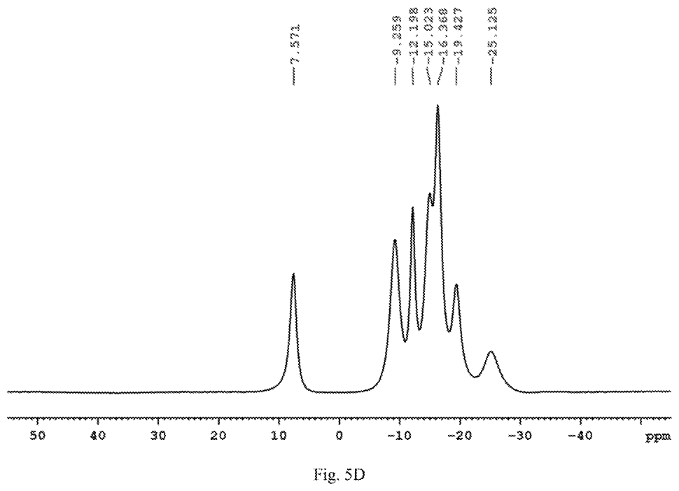

Azinostat. MS: Calculated m/z (M+H)⁺: 349.2690; Observed: 349.2958. FIGS. 5A-5D show ¹H, ¹³C, ¹¹B, and ¹¹B{¹H} NMR spectra of the product.

### Example 3A: Protein Binding Affinity Assay

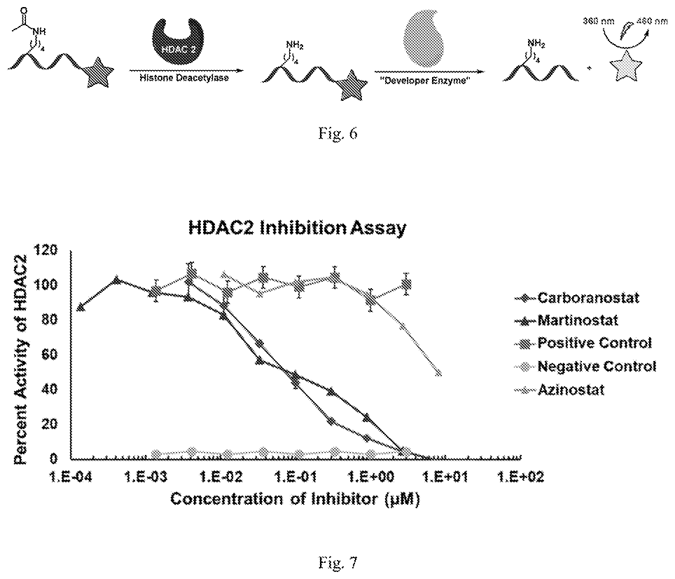

Fluorogenic HDAC Assay Kit (BPS: Bioscience, Catalog #: 50033, Size: 96 Reactions) This kit functions through an indirect measurement of HDAC activity. See FIG. 6.

First, the desired inhibitor is incubated with HDAC2 and a peptide substrate. The peptide substrate is a short peptide with an acetylated lysine incorporated in the sequence. The peptide substrate is also covalently attached to a fluorophore. The fluorophore is quenched a result of the covalent linkage to the peptide. HDAC2 can deacetylase the substrate, however, based on the potency and concentration of the inhibitor incubated with the enzyme, the deacetylation of substrate will be hindered. The deacetylation reaction continues for 30 minutes, after which the HDAC activity is fully abolished by addition of a known and potent HDAC2 inhibitor (Trichostatin A) at high concentrations. Then the “developer enzyme” solution is added. The “developer enzyme” selectively breaks the covalent linkage between the fluorophore and the deacetylated peptide. Specifically, the developer will not break the linkage between the fluorophore and an acetylated peptide (intact substrate). Thus, the concentration of the fluorophore released is directly proportional to HDAC activity during the deacetylation reaction. The higher fluorescence detected determines a higher HDAC activity, and as a result a lower inhibitor potency. Assessment of the fluorescence at various concentrations of a test inhibitor provides a measure for potency of the inhibitor.

Each assay is run in duplicate, fluorescence is measured in triplicate on a TECAN plate reader, each experiment is run in duplicate, and error is represented as standard deviation.

After receiving the kit, the TSA solution, HDAC substrate, HDAC assay buffer, and HADC developer are aliquoted. HDAC enzyme is diluted and aliquoted. Two BSA solutions were prepared. Before starting, the TSA solution, HDAC substrate, HDAC 2 enzyme, HDAC developer, and HDAC assay buffer were thawed on ice. The enzyme was stored diluted with 0.1% BSA solution. The following solutions were prepared after thawing.

BSA in H₂O Solution: A 1 mg/mL (0.1%) solution of BSA (bovine serum albumin) in H₂O was prepared by adding 5 mg of powdered BSA on top of 5 mL of milliQ water in a screw cap vial and leaving it at 4° C. to dissolve. After the BSA was dissolved, 4 mL of the prepared 0.1% BSA solution in water was used for either 20 aliquots of 75 μL (each aliquot meant for a 10 reaction set) or 20 aliquots of 125 μL (each aliquot meant for a 20 reaction set). The aliquots and the extra 1 mL BSA H₂O solution were stored at −80° C. 5 μL was used in all reactions. The 10 reaction sets use 50 μL; and 20 reaction sets use 100 μL.

BSA in HDAC Assay Buffer Solution: This solution was used to dilute HDAC enzyme and 5 μL was used in each blank control (10 μL per set). A 1 mg/mL (0.1%) solution of BSA in HDAC assay buffer was prepared by adding 2.5 mg of powdered BSA on top of 2.5 mL of HDAC assay buffer in a screw cap vial and leaving it at 4° C. to dissolve. 0.5 mL of this solution was divided into 15 μL aliquots for a total of 33 aliquots which were stored at −20° C. Slightly less than 2 mL of this solution was used to dilute the HDAC enzyme.

HDAC Assay Buffer Solution (HAB): 2.5 mL of the HDAC buffer was used for making the BSA buffer which dilutes the HDAC enzyme solution. The rest of the buffer solution was divided into aliquots that for dilution of TSA (10.8 μL per set), dilution of HDAC substrate (52.8 μL per 10 reaction set and 100.8 μL per 20 reaction set), buffering the reactions at 30 μL per reaction (i.e. making the master mixture with 330 μL per 10 reaction set or 630 μL per 20 reaction set), dilution of the test inhibitor and making inhibitor buffer at 5.4 μL per reaction (43.2 μL per 10 reaction set, 97.2 μL per 20 reaction set), dividing the HDAC assay buffer into 6 aliquots of 480 μL (each aliquot is for 10 reaction set) and 5 aliquots of 900 μL (each aliquot is for 20 reaction set), dividing the leftover solution into 50 μL aliquots and storing all the aliquots at −20° C.

HDAC Developer Solution (DEV): After the HDAC developer solution was thawed, the solution was divided into 2 aliquots of 630 μL (each aliquot is for a 10 reaction set) and 4 aliquots of 1150 μL (each aliquot is for a 20 reaction set). The leftover solution was divided into 60 μL aliquots and all the aliquots were stored at −80° C.

TSA Solution: When TSA solution was thawed, it was divided into 25 aliquots of 1.2 μL stock 200 μM solutions. The aliquots and the extra 70 μL of TSA solutions are stored at −80° C. Each aliquot has enough TSA for the 2 control inhibitor reactions for each set.

HDAC Substrate Solution: When HDAC substrate solution was thawed, it was divided into 9 aliquots of 2.2 μL (each aliquot was for a 10 reaction set) and 7 aliquots of 4.2 μL (each aliquot was for a 20 reaction set). The aliquots and the extra substrate solution were stored at −80° C. Each reaction used 0.2 μL substrate.

HDAC2 Enzyme Solution: 2 μg of enzyme was provided as a 2.73 mg/mL solution. The enzyme was stored at the 1 ng/μL concentration with 0.1% BSA solution in aliquots at −80° C. After the HDAC2 enzyme solution was thawed (and BSA solution was prepared), 1999.27 μL of 0.1% BSA solution was added to the stock HDAC solution. The 1 ng/μL solution was divided into 14 aliquots of 46 μL (each aliquot is for a 10 reaction set) and 14 aliquots of 96 μL (each aliquot is for a 20 reaction set). The aliquots and the extra 11 μL HDAC enzyme were stored at −80° C. Each reaction used 5 μL enzyme.

The 10 reaction sets were used to determine the proper concentration range of an inhibitor in order to calculate its IC50 value. The 20 reaction sets were used to measure seven different concentrations (range determined in a prior set) of inhibitor and calculate the IC50 value.

Inhibitors were diluted to 30 μM in DMSO and then serial diluted 1:3 six times for a range of seven concentrations. The following aliquot solutions were thawed on ice: diluted HDAC enzyme aliquot (based on reaction set), HDAC substrate aliquot (based on reaction set), TSA solution aliquot, HDAC developer aliquot (based on reaction set), HDAC assay buffer aliquot (based on reaction set), BSA in H₂O solution aliquot (based on reaction set), BSA in HDAC assay buffer solution aliquot. The assay components were combined as needed on the day of, and everything was kept on ice throughout the reaction set up. The inhibitor solutions were prepared as follows: 10.8 μL of the HDAC assay buffer was added to the 1.2 μL TSA solution aliquot and mixed. 21.6 μL of the HDAC assay buffer was added to 3.2 μL of DMSO and mixed. 10.8 μL of the HDAC assay buffer was added to 0.6 L of inhibitor solution test 1 and mixed. This was repeated for each inhibitor solution. The HDAC substrate was diluted by adding 52.8 μL of the HDAC assay buffer to the 2.2 μL HDAC substrate aliquot and mixing. In a 1.5-2 mL vial was prepared the master mixture with 55 μL of BSA solution in H₂O, 330 μL of the HDAC assay buffer, and 55 μL of diluted HDAC substrate solution. Each reaction was performed in duplicate. 40 μL of the master mixture was added in each well (20 or 40) of a black, low binding NUNC black microtiter plate. The inhibitor solutions were added as follows: 5 μL of the diluted DMSO in HDAC assay buffer solution was added to both of the blank control wells. 5 μL of the diluted DMSO in HDAC assay buffer solution was added to both of the positive control wells. 5 μL of the diluted TSA in HDAC assay buffer solution was added to both of the inhibitor control wells. 5 μL of the diluted inhibitor solution test 1 in HDAC assay buffer was added to both of the test 1 wells. That step was repeated for each of the remaining inhibitor solutions. The enzyme solutions were added as follows: 5 μL of the BSA in HDAC assay buffer solution was added to both of the blank control wells. 5 μL of the diluted enzyme solution in HDAC assay buffer was added to the rest of the wells. Incubate at 37° C. for 30 minutes. 50 μL of the HDAC assay developer solution was added to every well. Incubate at room temperature for 15 minutes. Samples were measured on a Tecan plate reader (excitation at 365 nm, detection at 450 nm) four times. The four readings and duplicates were averaged, and error bars represent standard deviation.

Assay data are shown in FIG. 7. The activity of HDAC is measured by a fluorescent assay, and a decrease in HDAC activity results in a decrease in fluorescence. The highly potent trichostatin A (TSA) was used as a negative control. The resulting IC50 values were calculated by fitting these curves to a sigmoidal fit. This preliminary data shows that Martinostat and Carboranostat have similar binding affinity for HDAC2.

### Example 3B

A human cell line will be incubated with inhibitors to test the decrease in acetylation throughout the cell as a means of measuring EC50.

### Example 4. Fluorescence Activated Cell Sorting (FACS)

Chinese hamster ovaries (CHO) cells gifted from the Sletten lab, were plated on 4 growth culture dishes. The dishes were incubated at 37° C., 5% CO₂ in FK12 medium with 20% and 10% Pen-Strep. After cells reached 90% confluency, the media was removed (4 plates), cells were washed with 5 mL of PBS each, then 1 mL trypsin-EDTA (0.25%) was added and cells were incubated for 2 minutes. 4 mL of media to each, and cells were spun down in falcon tubes at 4° C. After removing the media and resuspending in 10 mL of PBS, cells were counted on a hemocytometer with 2×10 μL. If the count was not at least 200, each of the four tubes was combined, spun down, PBS removed, resuspend, and repeated for a final wash. The cells were recounted on the hemocytometer. 250 cells were counted for the combined 4 plates at 2,500,000 cells/mL. To plate 200,000 cells per well 80 μL of cells suspended in 10 mL of media were added to each well followed by 2 μL treatment and 118 μL media for a total volume of 200 μL. Controls and treatments were performed in triplicate. Cells were incubated for 17-24 hours at 37° C., 5% C₀₂. In a V-bottom, clear 96-well plate 80 μL of cells was added to A1-3 Then cells were resuspended with a P1000 pipette and repeat for plate plan. 2 μL of inhibitor was added to their respective wells and the tip was changed every column. After adding 118 μL of media to every well using a reservoir the cells were resuspended with a P200 multichannel pipettor. Cells were incubated at 37° C. and 5% CO₂ for 18 hours. After incubation, the plate was spun down at 4° C., and washed with 150 uL FACS buffer (PBS with 6% FBS) twice. For cells killed with heat, they were transferred to FACS tubes after the first wash and heated at 90° C. in a water bath for 1 minute. These tubes were cooled to room temperature before adding propidium iodide. After the second wash, cells were resuspended in 150 μL FACS buffer and transferred to FACS tubes. 2 μL propidium iodide was added to each tube and 150 μL FACS was added to each tube for a total of 300 μL FACS buffer. The tubes were kept on ice for 15 minutes, and the flow cytometer measured fluorescence at 585 nm for 15,000 cells. Live and dead controls were used to calibrate the flow cytometer. Triplicate samples were averaged with the error bars representing standard deviation and results were reported as a percentage of live cells. The experiment was performed in duplicate.

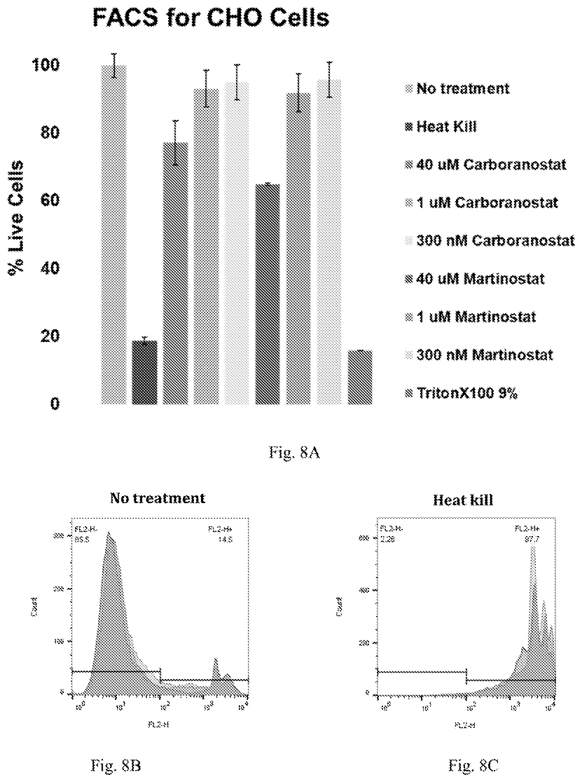

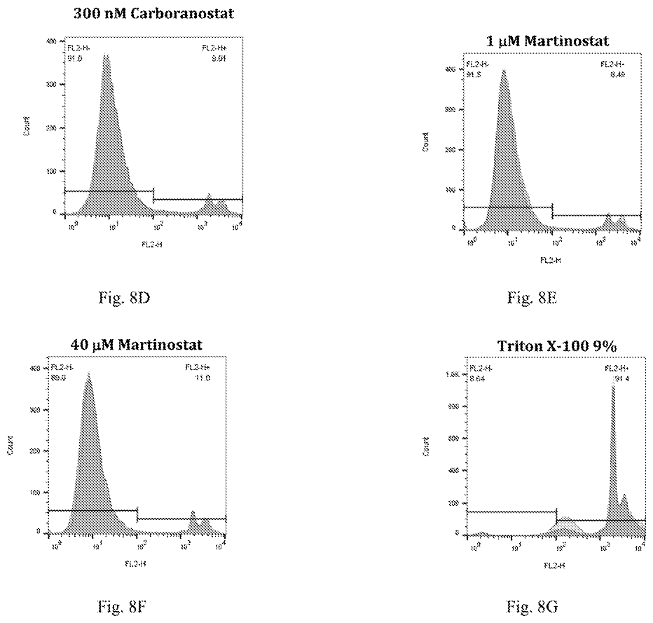

The data are shown in FIGS. 8A-8G. This FACS experiment measures the number of dead cells by detecting the fluorescence of propidium iodide. This data shows that at nM and μM concentrations, both inhibitors display similar activity and toxicity. The 300 nM inhibitor concentrations show low cytotoxicity. The 40 μM concentration is what would normally be administered to a murine animal for in vivo studies.

### Example 5A. MTS Assay

CellTiter 96® Aqueous Non-Radioactive Cell Proliferation Assay (Promega, Catalog #: G5421)

CHO cells were passaged and counted as previously described for FACS experiments. The solutions of MTS and PMS were thawed in a 37° C. bath. The 1 mL PMS solution was added to the 20 mL MTS solution and gently mixed (while protecting from light). The combined solution was aliquoted into Eppendorf tubes and wrap in foil (565 μL for 27 well experiments). Media was used as a blank with MTS/PMS, cells without treatment were used as a negative control, 9% Triton X-100 was used as a positive control, and 1 μL of inhibitor solutions were used at varying concentrations. Cells were plated at 10K and 20K cells/well in triplicate, resuspended with a P1000 pipet every column, inhibitors were added to their respective wells, and media was added from a reservoir using a P200 multichannel pipet, resuspending gently to give a final volume of 100 μL per well. Cells were incubated for 24 hours at 37° C., 5% CO₂. Following incubation, 9% Triton X-100 was added to cells for positive control and 20 μL of the MTS/PMS solution was added to each well for a final volume of 120 μL. Cells were incubated at 37° C. 5% CO₂ for 4 hours protected from light. Absorbance was measured at 490 nm using a Tecan plate reader four times. Readings and triplicates were averaged, and error bars represent standard deviation. The assay was also performed with primary mouse NPCs according to the protocol described above.

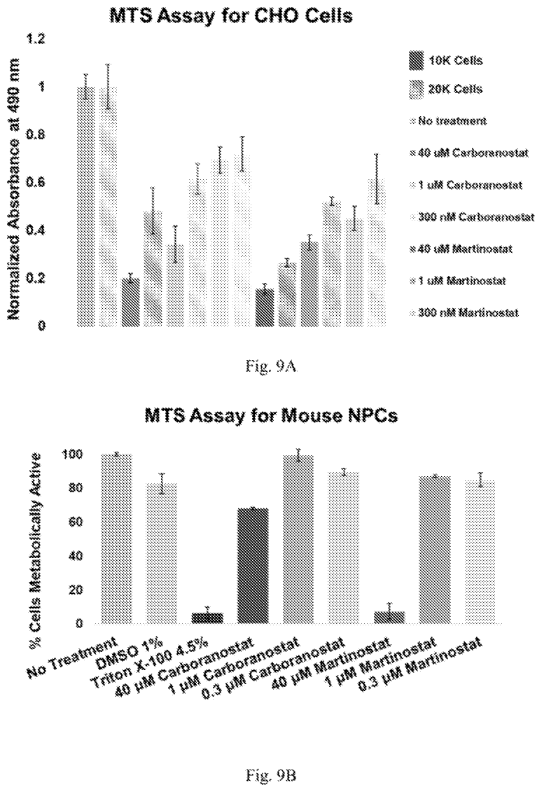

Data are shown in FIGS. 9A and 9B. This cell proliferation assay validates that the two inhibitors have similar toxicity. The perceived higher toxicity compared to the FACS data is a result of the MTS assay being both dependent on the number of cells at the start of the assay, but also on the development of the formazan, which has a time restriction since the assay reagents become cytotoxic themselves. This study still validates the low toxicity of both inhibitors.

### Example 5B

Blood-brain barrier permeability will be tested both by measuring the log P value as well as using a cellular-based model of the blood-brain barrier. ICP-AES and behavioral animal studies will also be carried out. Colorimetric binding studies used to measure the IC50 will be validated by binding studies using ITC.

### Example 6. TUNEL Assay

Click-iT® Plus TUNEL Assay (Molecular Probes Life Technologies, Catalog #: C10617)

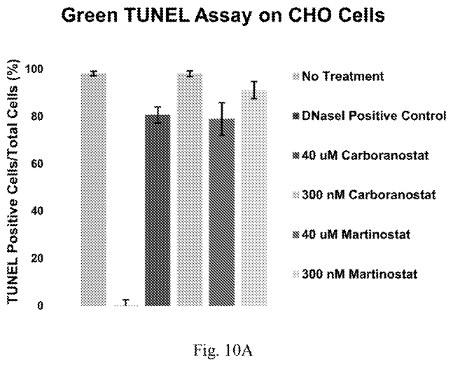

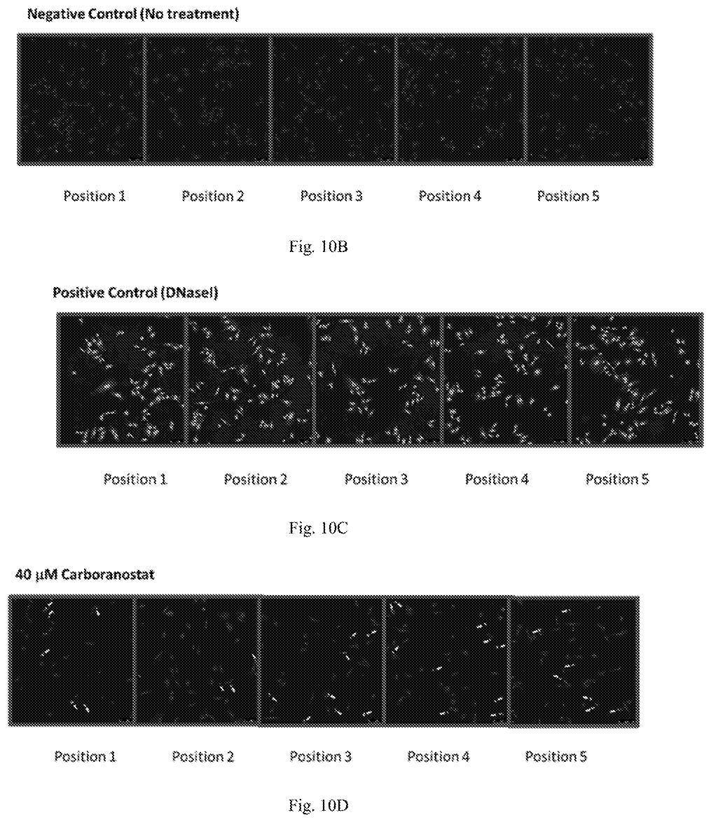

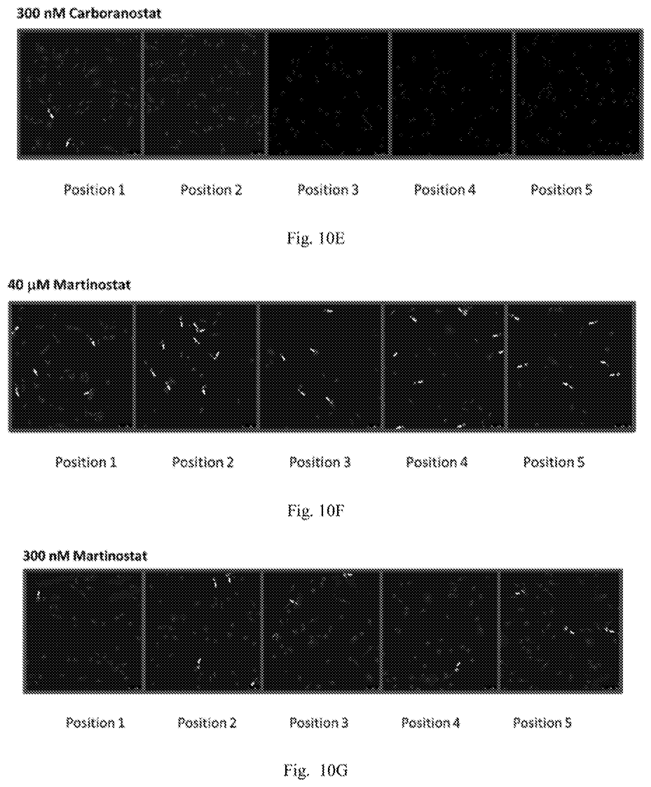

CHO cells were passaged and counted as previously described for FACS experiments. Cells without treatment were used as a negative control, DNaseI (Cat. no. 18068-015) was used to generate a positive control, and 10 μL of inhibitor solutions were used at varying concentrations. A 6-well plate was prepped with 22×22 mm coverslips in the wells. Coverslips were coated with FBS prior to plating the cells. Cells were plated at 300K cells/well, resuspend with a P1000, inhibitors were added to their respective wells, and media was added resuspending gently to give a final volume of 1 mL per well. Cells were incubated for 24 hours at 37° C., 5% CO₂, after which cells were between 60-70% confluency. Following incubation, the cells were fixed with 4% formaldehyde and permeabilized with 0.25% Triton X-100. Positive control cells were incubated with 2U DNaseI for 30 minutes at 37° C., 5% CO₂. For each well, the cells were treated according to the TUNEL assay protocol. Cell images were taken on a Leica confocal microscope at five positions per treatment. Cell counts were performed using ImageJ software. The assay was repeated twice for biological replicates, and error bars represent standard deviation. Assay data are shown in FIGS. 10A-10G.

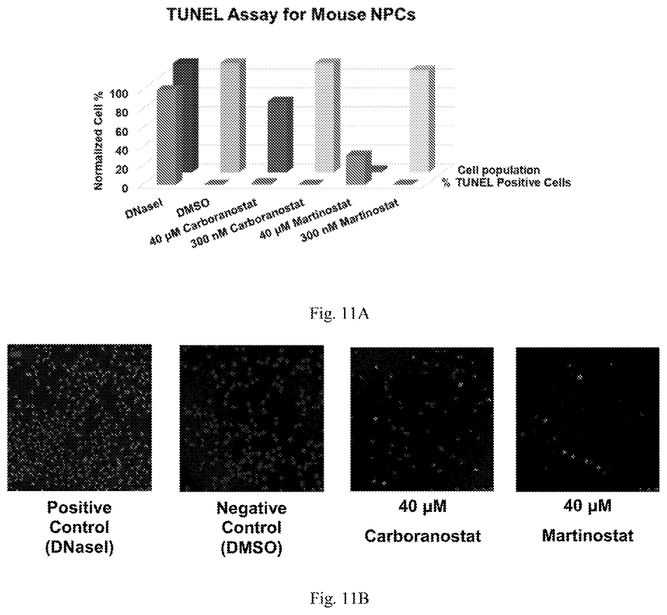

Primary mouse NPCs were passaged and counted as previously described. Cells without treatment were used as a negative control, DNaseI (Cat. no. 18068-015) was used to generate a positive control, and 10 μL of inhibitor solutions were used at varying concentrations. A 24-well plate was prepped with 12 mm circle glass coverslips in the wells. Coverslips were coated with poly-L-lysine followed by laminin prior to plating the cells. Cells were plated at 50K cells/well, and then the plate was centrifuged at 233 g (1000 rpm) for 3 minutes, low acceleration, low deceleration to allow for cells to attach to the coated slips in minimum media. For inhibitor wells, DMSO or inhibitor was added to the well after 30 minutes of incubation with a final DMSO concentration of <1%. Plates were incubated at 37° C., 5% CO₂ for 18-24 hours or until cells reached between 60-70% confluency. Final well volume was 0.5 mL per well. Following incubation, the cells were fixed with 4% formaldehyde and permeabilized with 0.25% Triton X-100. Positive control cells were incubated with 2U DNaseI for 30 minutes at 37° C., 5% CO₂. For each well, the cells were treated according to the TUNEL assay protocol. Cell images were taken on a Leica confocal microscope at five positions per treatment. Cell counts were performed using ImageJ software. The assay was repeated twice for biological replicates, and error bars represent standard deviation. Assay data are shown in FIG. 11A. Representative confocal images are shown in FIG. 11B. Note that the perceived elevation in TUNEL positive cells for 40 μM Martinostat is due to an observed higher toxicity as shown by the average cell population count from the 5 positions of the confocal images.

### Example 7. Proposed Syntheses of Carboranostat Derivatives

To generate a library of Carboranostat derivatives that vary in their orientation and strength of the cluster's dipole, as well as in the sterics and electronics of the vertex substituents. The proposed syntheses below take advantage of the cage-walking mechanism for the Pd-catalyzed cross-coupling along with multifunctionalization and C-vertex functionalization.

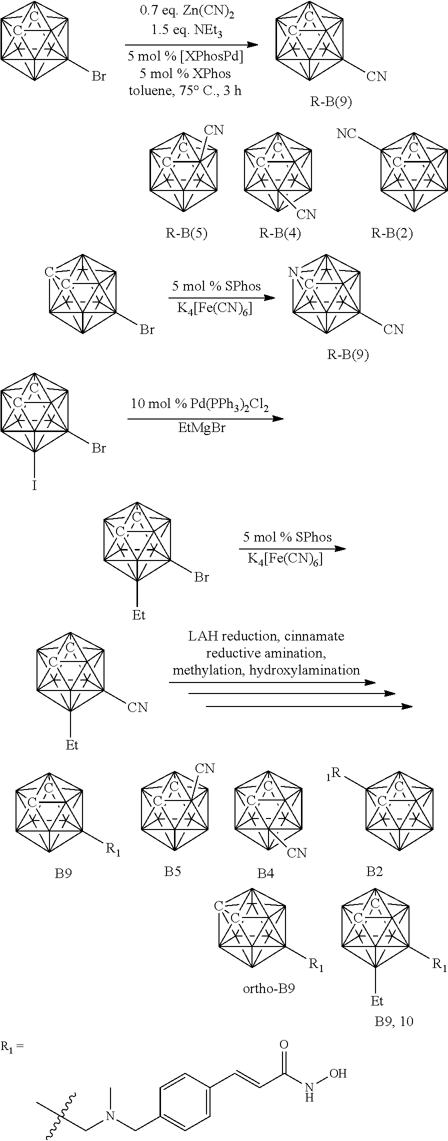

### Example 8

Inhibitors will be used to study behavior in cocaine addicted rats, and studies will include dose-response and behavioral studies in rats. Dose-response studies will involve three doses and three rats/dose at concentrations of 6.9 mM, 17.3 mM, and 31.1 mM as determined by in vitro studies and common practice. The biodistribution and accumulation of Carboranostat in these rats will be analyzed following euthanization, cryo-sectioning the tissue, and performing ICP-AES on the tissue. The behavioral studies will monitor the prevention and rescue through cocaine-addiction simulation developed in the Kennedy lab (reference).

### Example 9

Neural degeneracy in general can be studied using the known homocysteic acid (HCA) assay, in which inhibitors of class I, IIa, IIb, and IV will be tested for their ability to rescue cortical neurons during induced oxidative stress conditions. This assay can be performed as previously described. (Butler, K. V.; Kalin, J.; Brochier, C.; Vistoli, G.; Langley, B.; Kozikowski, A. P. Rational Design and Simple Chemistry Yield a Superior, Neuroprotective HDAC6 Inhibitor, Tubastatin A. JACS 2010, 132, 10842-10846.)

### Example 10

Inhibitors will also be studied for use as anti-cancer drugs in treating glioblastoma (GBM). In vitro studies will involve testing cell survival, proliferation, sphere formation, and migration of GBM cells as well as normal progenitor and mature neural cells. (Sawa, H.; Murakami, H.; Kumagai, M.; Nakasato, M.; Yamauchi, S.; Matsuyama, N.; Tamura, Y.; Satone, A.; Ide, W.; Hashimoto, I.; Kamada, H. Histone Deacetylase Inhibitor, FK228, Induces Apoptosis and Suppresses Cell Proliferation of Human Glioblastoma Cells in Vitro and in Vivo. Acta. Neuropathol. 2004, 107, 523-531; Hong, X.; Chedid, K.; Kalkanis, S. N. Glioblastoma Cell Line-Derived Spheres in Serum-Containing Medium Versus Serum-Free Medium: A Comparison of Cancer Stem Cell Properties. Int. J. Oncol. 2012, 41, 1693-1700.). In vivo studies will include xenografted mice and testing the HDAC inhibitor against tumor growth. (Visnyei, K.; Onoder, H.; Damoiseaux, R.; Saigusa, K.; Petrosyan, S.; Vries, D. D.; Ferrari, D.; Saxe, J.; Panosyan, E. H.; Masterman-Smith, M.; Mottahedeh, J.; Bradly, K. A.; Huang, J.; Sabatti, C.; Nakano, I.; Kornblum, H. I. A Molecular Screening Approach to Identify and Characterize Inhibitors of Glioblastoma Stem Cells. Mol. Cancer Ther. 2011, 10, 1818-1828).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

We claim:

1. A compound having the structure of Formula (I) 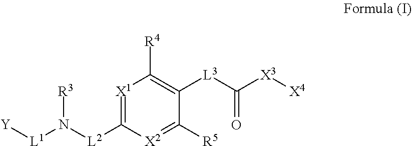
or a pharmaceutically acceptable salt, ester, or prodrug thereof, wherein
Y is optionally substituted boron cluster (e.g., an icosahedral boron cluster);
R³ is H or optionally substituted alkyl;
R⁴ is selected from H, halo, hydroxyl, and optionally substituted alkyl;
R⁵ is selected from H, halo, hydroxyl, and optionally substituted alkyl;
each of L¹, L², and L³ is independently selected from a bond, optionally substituted alkylene, and optionally substituted alkenylene;
X¹ is CR⁶ or N;
X² is CR⁷ or N;
each R⁶ and R⁷ is independently selected from H, halo, hydroxyl, and optionally substituted alkyl;
X³ is selected from —NH—, —N(OH)—, and O; and
X⁴ is selected from H, optionally substituted alkyl, and optionally substituted aryl.

2. The compound of claim 1, wherein the boron cluster is a B₁₂H₁₂, B₁₀H₁₀, or B₆H₆ cluster, or a carborane.

3. The compound of claim 1 or claim 2, wherein the boron cluster is a B₁₂H₁₂, B₁₀H₁₀, or B₆H₆ cluster.

4. The compound of claim 1 or claim 2, wherein the boron cluster is a carborane.

5. The compound of claim 4, wherein the carborane is a C₂B₁₀ carborane.

6. The compound of claim 4 or 5, wherein the carborane is an icosahedral closo-carborane.

7. The compound of any one of claims 1 to 6, wherein Y is selected from 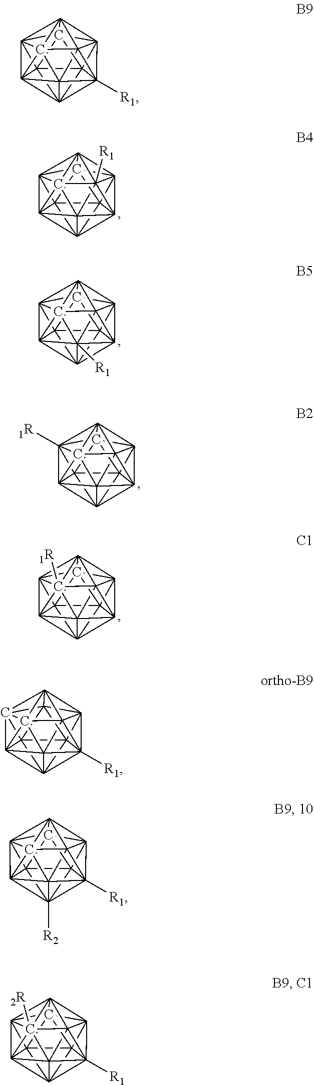
or a pharmaceutically acceptable salt thereof,
wherein
the unlabeled atoms of the icosahedron are boron;
R¹ represents a bond to L¹; and
R² is selected from halo, cyano, optionally substituted alkyl, optionally substituted amine, and —OR⁸, wherein R⁸ is optionally substituted alkyl or a protecting group.

8. The compound of any one of the preceding claims, wherein R³ is alkyl or haloalkyl.

9. The compound of claim 8, wherein R³ is alkyl, preferably methyl.

10. The compound of any one of the preceding claims, wherein each of R⁴, R⁵, R⁶, and R⁷ is independently selected from H, halo, hydroxyl, methyl, and —CF₃.

11. The compound of any one of the preceding claims, wherein each of R⁴, R⁵, R⁶, and R⁷ is independently selected from H, —F, —Cl, and —Br.

12. The compound of any one of the preceding claims, wherein R⁴, R⁵, R⁶, and R⁷ are each H.

13. The compound of any one of the preceding claims, wherein L¹ and L² are each independently alkylene.

14. The compound of claim 13, wherein L¹ and L² are each independently —CH₂—.

15. The compound of any one of the preceding claims, wherein L³ is alkenylene.

16. The compound of claim 15, wherein L³ is —CH═CH—.

17. The compound of any one of claims 1 to 14, wherein L³ is a bond.

18. The compound of any one of the preceding claims, wherein X¹ is CR⁶.

19. The compound of claim 18, wherein R⁶ is H.

20. The compound of any one of the preceding claims, wherein X² is CR⁷.

21. The compound of claim 20, wherein R⁷ is H.

22. The compound of any one of the preceding claims, wherein X³ is N(OH)—.

23. The compound of any one of the preceding claims, wherein X⁴ is H.

24. The compound of any one of claims 1 to 7, wherein the compound has the structure:

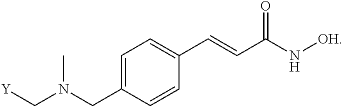

25. The compound of claim 1 wherein the compound is 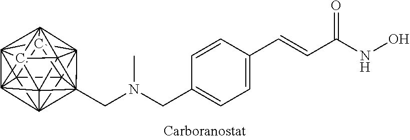
or a pharmaceutically acceptable salt thereof.

26. The compound of any one of claims 1 to 7, wherein the compound has the structure:

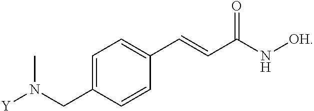

27. The compound of claim 1, wherein the compound is 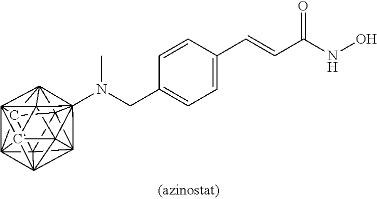
or a pharmaceutically acceptable salt thereof.

28. A pharmaceutical composition comprising a compound of any one of the preceding claims and a pharmaceutically acceptable excipient.

29. A method of inhibiting an histone deacetylase (HDAC) enzyme in a subject, comprising administering to the subject a compound or composition of any one of claims 1-28.

30. A method of treating a disease selected from cancer, a disease of the central nervous system, and an inflammatory autoimmune disease in a subject, comprising administering to the subject a compound or composition of any one of claims 1-28.

31. The method of claim 30, wherein the disease is cancer.

32. The method of claim 31, wherein the cancer is selected from glioma, e.g., glioblastoma; hematological cancer, e.g., leukemia or lymphoma; and non-small cell lung cancer.

33. The method of claim 30, wherein the disease is a disease of the central nervous system.

34. The method of claim 33, wherein the disease of the central nervous system is selected from mood and mental disorders, e.g., depression, schizophrenia, or bipolar disorder; neurodegenerative diseases, e.g., Huntington's disease, or Alzheimer's disease; drug addiction, e.g., cocaine addiction; and disorders of learning, memory, or cognition.

35. The method of claim 30, wherein the disease is an inflammatory autoimmune disease.

36. A process for preparing a compound of Formula (I) 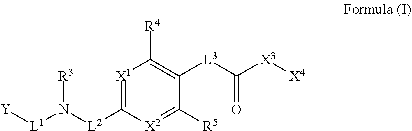
comprising:
preparing a compound of Formula (II) by reacting a compound of Formula (III) with a compound of Formula (IV) under reductive alkylation conditions: [chemical structure]
